SEQUENCE LISTING (8 sequences)

SEQ ID NO: 1 - DNA, 25 bp, Artificial Sequence
  Synthetic sequence
agtaaggtttttttttaacaatagg

SEQ ID NO: 2 - DNA, 28 bp, Artificial Sequence
  Synthetic sequence
caataaaatcattctaaaaaataacaac

SEQ ID NO: 3 - DNA, 20 bp, Artificial Sequence
  Synthetic sequence
gaaggtgtattgaagtgtgg

SEQ ID NO: 4 - DNA, 21 bp, Artificial Sequence
  Synthetic sequence
ccaaaaaaaaaacccaaccca

SEQ ID NO: 5 - DNA, 27 bp, Artificial Sequence
  Synthetic sequence
gtgtaagttattgtgattctgattgtg

SEQ ID NO: 6 - DNA, 30 bp, Artificial Sequence
  Synthetic sequence
ctttaactaccctctctaaattaaaaaacc

SEQ ID NO: 7 - DNA, 23 bp, Artificial Sequence
  Synthetic sequence
ggtttttggatagagtggggata

SEQ ID NO: 8 - DNA, 25 bp, Artificial Sequence
  Synthetic sequence
caaaaactaccaaacvcatcaaacc

(19) United States
(12) Patent Application Publication
(10) Pub. No.: US 2022/0136051 A1
(43) Pub. Date: May 5, 2022

(54) METHODS FOR IDENTIFYING AND IMPROVING T CELL MULTIPOTENCY

(71) Applicant: St. Jude Children's Research Hospital, Memphis, TN (US)

(72) Inventors: Yiping Fan, Memphis, TN (US); Jeremy Crawford, Memphis, TN (US); Benjamin Youngblood, Memphis, TN (US)

(73) Assignee: St. Jude Children's Research Hospital, Memphis, TN (US)

(21) Appl. No.: 17/432,721

(22) PCT Filed: Feb. 24, 2020
(86) PCT No.: PCT/IB2020/051567

§ 371 Date: Aug. 20, 2021

Related U.S. Application Data
(60) Provisional application No. 62/809,034, filed on Feb. 22, 2019.

(51) Int. Cl. C12Q 1/6881; G16B 40/20

(52) U.S. Cl. CPC C12Q 1/6881; C12Q 2600/154; G16B 40/20

(57) ABSTRACT

Provided herein are methods and compositions for determining T-cell differentiation by comparing the methylation status of T cells relative to a T cell methylation index and using this determination to identify or isolate populations of T cells having desired T cell multipotency. Further, the present methods and compositions can be used to monitor or treat symptoms of chronic infections, autoimmune diseases, and cancer.

### STATEMENT OF FEDERALLY SPONSORED RESEARCH OR DEVELOPMENT

This invention was made with government support under grants AI114442 and AI109565 awarded by the National Institutes of Health. The government has certain rights in the invention.

### FIELD OF THE INVENTION

The invention relates to the field of cell biology and immunology. In particular, the invention relates to a method for detecting T-cell differentiation potential based on DNA methylation status, methods for isolating T-cells at a specific stage of differentiation, and compositions including said T-cells. The present methods and compositions can be used to monitor or treat symptoms of chronic infections, autoimmune diseases, and cancer.

### BACKGROUND OF THE INVENTION

Following an infection, naïve CD8 T cells are stimulated by dendritic cells (DC) displaying pathogen-derived peptides on MHC class I molecules (signal 1) and costimulatory molecules (signal 2). Additionally, pathogen-induced inflammatory cytokines also act directly on the responding CD8 T cells to regulate their expansion and differentiation.

Flow cytometry based-analyses are typically used to define the differentiation status of T cells based on the expression of various surface markers. However, the phenotypic markers used to delineate the differentiation status of T cells are significantly altered during T cell product generation and cannot be used to infer the differentiation status. Therefore, there is need in the art for improved methods to identify or isolate T cells at a given differentiation stage.

### SUMMARY OF THE INVENTION

Epigenetic modifications, such as DNA methylation play an important role in reinforcing the differentiation status of T cells. As provided herein, analysis of DNA methylation can provide a stable and accurate marker for assessing T cell differentiation. Accordingly, provided herein are methods of identifying the differentiation stage of T cells based on DNA methylation status of the genome of the subject T cell. These methods further enable the identification and isolation of T cells with enhanced multipotency, which can be useful in a variety of therapeutic methods and compositions. For example, the methods and compositions can be used to treat symptoms of chronic infections, autoimmune diseases, and cancer. Further, the methods and compositions encompass predicting T-cell activity by measuring the methylation status of specific CpG sites in the genome, comparing the methylation status to a multipotency index to identify and separate populations of CD8 T cell having desired T cell differentiation states. The methods herein can further be used to monitor subjects with chronic infections, autoimmune diseases, or cancer that would benefit from personalized therapy.

### BRIEF DESCRIPTION OF THE DRAWINGS

FIGS. 1A-1J: Generation of the human CD8 T cell DNA methylation landscape for assessment of beta cell-specific CD8 T cell differentiation (FIG. 1A) Representative FACS plots showing PD-1 expression among true naïve and memory CD8 T cells subsets from healthy adult subjects (Upper panel) and cell surface expression of CCR7, CD45RO, and PD-1 among HIV-specific CD8 T cells from HIV-ART patients (Lower Panel). (FIG. 1B) Graphs depicting PCA of methylation status of the total CpG sites in CD8 T cells showing 63.5% of principal component 1 (PC1). Principal component 2=4% of variance. Naïve CD8 T cells from HIV patients (n=3), T1D patient (n=5), and healthy adult donors [HD] (n=4), T1D Tetramer+(n=5), HD Tscm (n=3), Tcm (n=3), Tem (n=3), and HIV Tetramer+(n=4). (FIG. 1C) Representative FACS plots showing Tetramer staining for beta cell-specific CD8 T cells from T1D patients as well as CCR7 and CD45RA cell surface expression among total and T1D Tetramer positive CD8 T cells (Upper panel). Table showing disease-associated features of T1D patients used for isolating self-reactive CD8 T cells from peripheral blood (Lower panel). (FIG. 1D) Summary graph of the total number of DMRs in T1D-specific CD8 T cells genomes relative to naïve CD8 T cell genome. The number of demethylated regions was calculated based on > or =30% methylation difference between the two cell populations. The number of methylated regions was calculated based on < or =−30% methylation difference between the two populations (Lower panel). Pie chart showing the percentage of DMRs across the genome of T1D-specific CD8 T cells relative to naïve CD8 T cells (Upper panel). (FIG. 1E) Venn-diagram showing the number of DMRs in T1D-specific CD8 T cells genomes relative to Tcm, Tem, Tscm, and HIV-specific CD8 T cell genome. The number of demethylated regions was calculated based on > or =30% methylation between the two population. The number of methylated regions was calculated based on < or =−30% methylation between the two population. (FIG. 1F) Upper panel provides pie chart showing the percentage of DMRs across the genome of T1D-specific CD8 T cells relative to Tcm, Tem, Tscm, and HIV-specific CD8 T cell genome. Lower panel provides summary graph of the number of DMRs in T1D-specific CD8 T cells genomes relative to Tcm, Tem, Tscm, and HIV-specific CD8 T cell genome. The number of demethylated regions was calculated based on > or =30% methylation between the two population. The number of methylated regions was calculated based on < or =−30% methylation between the two population. (FIG. 1G) Normalized plots of CpG methylation at sites surrounding and within DMRs of sternness programs including transcription factors (BATF and TOX), and the de novo DNA methyl transferase enzyme (DNMT3A). Red and blue lines depict methylated and unmethylated CpG sites, respectively. (FIG. 111) Venn-diagram showing unique and overlapping DMRs in Tem and Tcm CD8 T cells relative to T1D Tetramer+CD8 T cells. DMR calculations were defined based on pairwise comparisons from the total of all replicate samples. (FIG. 1I) Normalized plots of CpG methylation at sites surrounding and within DMRs of sternness programs including chemokine receptor (CCR5), CCL5 and E3 ubiquitin ligase (FBXO10). Red and blue lines depict methylation and demethylation of CpG sites, respectively. (FIG. 1J) Venn-diagrams showing unique and overlapping DMRs in T1D-specific and Tem CD8 T cells relative to naive CD8 T cells and in T1D-specific and Tscm CD8 T cells relative to TEM CD8 T cells.

FIG. 2: DMR-associated genes among beta-cell specific CD8 T cells and HIV-specific CD8 T cells were cross-referenced with the previously published genes targeted for de novo DNA methylation during murine T cell exhaustion. De novo DNA methylation programs were defined as the genes absent in Dnmt3a deficient CD8 T cells isolated from chronically infected mice.

FIGS. 3A-3C: Demonstrates that novel human multipotency index predicts beta cell-specific CD8 T cells to retain a degree of developmental plasticity comparable to Tscm (FIG. 3A) Heatmap analysis of methylation status for 45 CpG sites among our T cell methylome datasets based on sternness index. (FIG. 3B) Heat map showing the methylation status of 245 CpG sites of HIV-specific, T1D-specific, naïve, Tem, Tcm, and Tscm CD8 T cells used to define the human T cell multipotency index. The CpG sites were identified from a machine learning algorithm using naïve and HIV-specific CD8 T cells as the training data sets. Red and blue intensity depict methylated and unmethylated CpG sites. (FIG. 3C) A normalized human T cell multipotency score (0-1) was generated as described in methods based on the newly identified CpG sites that delineate the developmental potential of human T cells. Weight assignment for the methylation status at the 245 CpG sites is provided in Table 1. The normalized multipotency index was applied to T1D-specific, Tem, Tcm, and Tscm CD8 T cell methylomes.

FIG. 4: Self-reactive human CD8 T cells acquire effector associated epigenetic programs. Normalized plots of CpG methylation at sites surrounding and within DMRs of effector molecules (PRF1, GZMK, and IFNg) and transcription factors (Eomes, TBX21 and Tcf7) obtained from WGBS analysis. Red and blue lines depict methylated and unmethylated CpG sites, respectively.

FIGS. 5A-5F: Single cell ATACseq profiling identifies naïve and effector epigenetic programming within individual beta cell-specific CD8 T cells (FIG. 5A) TSNE analysis of individual cell chromatin accessibility profiles for all (aggregated) Tetramer+CD8 T cells and representative naïve and Tem populations from three donors. (FIG. 5B) Heatmap for the chromatin accessibility profile of CCR7 for all cells. (FIG. 5C) Heatmap for the chromatin accessibility profile of LEF1 and DNMT3a for all cells. (FIG. 5D) Heatmap for the chromatin accessibility profile of TOX and TBX21 for all cells. (FIG. 5E) Heatmap for the chromatin accessibility profile of IFNG, PRF1 and GZMK for all cells. (FIG. 5F) Composite ATACseq profiles showing accessibility peaks across the loci of LEF1, TCF7, DNMT3a, TBX21, IFNg and PRF1 loci for naïve, Tem, and beta cell-specific CD8 T cells (Tetramer+).

FIGS. 6A-6B: Stemness-associated DNA methylation programs are maintained during in vitro antigen-driven proliferation of human T1D-specific CD8 T cells. (FIG. 6A) Experimental setup for in vitro stimulation of total PBMCs from T1D patients. PBMCs were labeled with cell proliferation dye (cell trace violet-CTV) and subsequently maintained in culture in the presence of mixture of peptides specific for T1D-specific CD8 T cells for 14-24 days. Phenotypic analyses of beta cell-specific CD8 T cells prior to and after 14-24 days of peptide stimulation. Antigen-driven expansion of beta-cell specific CD8 T cells results in down regulation of CCR7 and upregulation of CD95. (FIG. 6B) Representative bisulfite sequencing analysis and bar graph showing % CpG methylation for individual CpG sites of sternness programs DNMT3A (CpG sites 12-18 shown) and TOX (CpG sites 4-10 shown) from beta cell-specific CD8 T cells post peptide stimulation relative to the methylation status of bona fide effector memory CD8 T cells.

FIGS. 7A-7C: Lymphoid-homing murine beta cell-specific CD8 T cells retain phenotypic and epigenetic programs indicating developmental plasticity. (FIG. 7A) Representative FACS plots showing the phenotype of tetramer positive beta cell-specific CD8 T cells isolated from murine spleen, pancreatic lymph node, and pancreas. Phenotypic characterization includes cell surface expression of CD44, PD-1, CD127, and CD62L. (FIG. 7B) Summary graph from three different mice showing cell surface expression of CD44, PD-1, CD127, and CD62L on tetramer positive beta cell-specific CD8 T cells isolated from spleen, pancreatic lymph node, and pancreas (n=3). (FIG. 7C) Normalized plots of CpG methylation at sites surrounding and within DMRs of effector molecules (Ifng, Gzmk) and transcription factors (Tcf7, Batf, Eomes, and TBX21) obtained from WGBS analysis. Red and blue lines depict methylated and unmethylated CpG sites, respectively.

FIGS. 8A-8B: Novel murine multipotency index predicts terminal differentiation of beta cell-specific CD8 T cells isolated from the pancreas (FIG. 8A) Heat map showing the methylation status of 177 CpG sites of exhausted, lymphatic-derived, pancreas-derived, and naive CD8 T cells used to define the murine T cell multipotency index. The CpG sites were identified from a machine learning algorithm using naïve and LCMV-specific exhausted CD8 T cells as the training data sets. Red and blue intensity depict methylated and unmethylated CpG sites, respectively. (FIG. 8B) A murine T cell multipotency score was generated as described in Methods based on newly identified CpG sites that delineate the developmental potential of murine T cells. The multipotency score was derived from a machine learning algorithm using naïve and LCMV-specific exhausted CD8 T cell methylomes as the training datasets. LCMV-specific CD8 T cell methylomes were used as test datasets for the multipotency index. The multipotency score was based on weight assignment for methylation status at 177 CpG sites.

FIGS. 9A-9F: Stemness-associated DNA methylation programs persist during low-affinity TCR stimulation in LCMV-specific CD8 T cells. (FIG. 9A) Experimental setup showing adoptive transfer of CD45.1+P14 cells into WT B6 CD45.2 mice. One day later, mice were infected with either acute LCMV (Armstrong), chronic LCMV (CL13) containing high affinity gp33 epitope, chronic LCMV (C6) containing low affinity gp33 epitope. Four and eight weeks later, gp33-specific CD8 T cells (P14) were FACS purified for phenotypic and DNA methylation analysis. (FIG. 9B) Histograms showing PD-1 and KLRG-1 expression in adoptively transferred CD45.1 P14 T cells 4 and 8 weeks post infection (p.i) using acute, high affinity chronic CL13, or low affinity chronic CL13 (C6). Open lines represent CD45.1 cell population while shaded area represents CD45.2 cell population (endogenous response) (FIG. 9C) MFI of PD-1 expression in adoptively transferred CD45.1 P14 T cells 8 weeks post infection (p.i) from mice infected with the acute strain of LCMV, the high affinity chronic CL13 strain, or low affinity chronic C6 strain. (FIG. 9D) Chromosomal location of TOX locus. (FIG. 9E) Box and Whisker graph showing minimum and maximum values of CpG methylation of TOX locus in Naïve CD45.1 P14 cells from uninfected mice and CD45.1 P14 cells from Armstrong, high affinity CL13, and low affinity CL13 infected mice (Week 4 and Week 8 post infection) (means+/−SEM) at each site of the TOX locus (n=3-7) location. Dotted line was drawn to connect between the average among the different groups. (FIG. 9F) Representative bisulfite sequencing analysis of TOX DMR in CD45.1 P14 cells 4 weeks post infection using high affinity chronic CL13, or low affinity chronic CL13 (C6). Open circles represent unmethylated CpG while closed circles represent closed CpG.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods are provided herein for identifying the stage of differentiation of T-cells (e.g., CD8 T cells) based on the methylation status of the T cells. Specifically, the method involves detecting the methylation status of the subject T cells (e.g., CD8 T cells) and comparing the methylation status to a multipotency index to determine the stage of differentiation, and accordingly the multipotency or stemness, of the T cells. As used herein, the term “multipotency”, “differentiation potential”, or “stemness” refers to the potential of a T cell to further differentiate.

Following exposure to antigen, naïve T cells undergo proliferation and differentiation into memory T-cell subsets, eventually forming terminally differentiated effector T cells. As T cells mature, they acquire effector functions and lose the ability for self-renewal and survival. A minority will survive the contraction phase and become long-lived memory T cells, which have the ability to acquire effector functions upon reinfection. Memory T cells have been characterized by their phenotypic and functional profiles into T-cell subsets, typically stem memory (SCM), central memory (CM), and effector memory (EM) T cells. The multipotency of a T cell corresponds to its stage of differentiation, with naïve T cells having the highest multipotency and long-lived T cells (e.g., effector memory T cells) having a lower multipotency compared to other T cell subsets.

The methods and compositions disclosed herein utilize the methylation status of a particular genomic locus or combination of loci to determine the stage of differentiation, and the corresponding multipotency, of a T cell (e.g., CD8 T cell). The term “methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively. By “hypermethylation” or “increased methylation” is meant an increase in methylation of a region of DNA (e.g., a genomic locus as disclosed herein) that is considered statistically significant over levels of a control population. “Hypermethylation” or “increased methylation” may refer to increased levels seen in a subject over time or can refer to the methylation level relative to the methylation status of the same locus in a naïve T cell.

Moreover, the stage of differentiation, and the corresponding multipotency, of T cells (e.g., CD8 T cells) can be predicted based on measuring the methylation status of one or more than one genomic locus (e.g., one or more CpG sites, such as the CpG sites described in Table 1) and comparing the methylation status to a methylation index. Accordingly, a “methylation profile” or “methylation status” refers to a set of data representing the methylation states or levels of one or more loci within a molecule of DNA from e.g., the genome of an individual or cells or sample from an individual. The profile can indicate the methylation state of every base in an individual, can comprise information regarding a subset of the base pairs (e.g., the methylation state of specific restriction enzyme recognition sequence) in a genome, or can comprise information regarding regional methylation density of each locus. In some embodiments, a methylation profile refers to the methylation states or levels of one or more genomic loci (e.g., CpG sites) described herein. In more specific embodiments, a methylation profile refers to the methylation status of a gene, promoter, transcription factor, 3′ untranslated region (UTR), or regulator of cellular proliferation. From the methylation status of a T cell (e.g., a CD8 T cell), a multipotency score can be established that identifies the differentiation stage of the T cell. This multipotency score can further aid in isolation of desired T cells (e.g., CD8 T cells) for use in therapeutic compositions and methods, as further described herein.

### II. Methods of Identifying T-Cell Differentiation Stage

Compositions and methods are provided herein for identifying the stage of differentiation of T-cells by detecting the methylation status of a plurality of CpG sites in subject T cells (e.g., CD8 T cells) and comparing the methylation status to a multipotency index. A multipotency score can be generated based on this comparison that is indicative of the differential potential (i.e., multipotency) of the subject T cells. These methods are further useful for isolating cells at a desired stage of differentiation.

The multipotency index can be established using one or more machine learning algorithms (e.g., a supervised analysis) of genome-wide methylation in T cells (e.g., CD8 T cells, such as human CD8 T cells) at varying stages of differentiation. Machine learning analyses (e.g., supervised analysis) of other cell types are described, for example, in Malta et al, 2018, Cell, 173, 338-354, which is hereby incorporated by reference. For example, based on a supervised analysis of T cells (e.g., CD8 T cells), particular methylation sites (e.g., one or more CpG sites, such as the CpG sites described in Table 1) that are predictive of T cell differentiation potential can be identified. For example, a supervised analysis can be used to identify one or more CpG sites (e.g., one or more CpG sites, such as the CpG sites described in Table 1) that are hypomethylated in naïve T cells (e.g., CD8 T cells) compared to antigen-specific T cells (e.g., HIV-specific CD8 T cells), thereby indicating that the methylation sites of interest are differentially methylated in different subsets of T cells. As used herein, “hypomethylated” or “decreased methylation” is meant a decrease in methylation of a region of DNA (e.g., a genomic locus as disclosed herein) that is considered statistically significant relative to levels of a control population. “Hypomethylation” or “decreased methylation” may also refer to decreased levels seen in a subject over time or can refer to the methylation level relative to the methylation status of the same locus in a T cell of a different subset or stage of differentiation.

Methylation at the genomic sites of interest (e.g., one or more CpG sites, such as the CpG sites described in Table 1) can then be selected and assessed via machine learning methods to calculate a weighted score for each site that indicates the degree to which the particular site is predictive of differentiation status. Training sets based on the methylation status of one or more methylation sites (e.g., one or more CpG sites, such as the CpG sites described in Table 1) can be used as input to a machine learning algorithm to calculate a multipotency signature or index. A variety of machine learning methods are known in the art (see, e.g., Malta et al, 2018, Cell, 173, 338-354) and can be applied to the present methods. For example, in certain instances, one-class logistic regression can be used to obtain the multipotency index. Further examples of widely used machine learning methods, algorithms, computer programs, or systems that can be applied herein include, but are not limited to, a Neural network (multi-layer perceptron), Support vector machines, k-nearest neighbors, Gaussian mixture model, Gaussian, naive Bayes, Decision tree, and RBF classifier. In some embodiments, the multipotency index is generated using Linear classifiers (for e.g., partial least squares determinant analysis (PLS-DA), Fisher's linear discriminant, Logistic regression (e.g., one-class logistic regression), Naive Bayes classifier, Perceptron), Support vector machines (for e.g., least squares support vector machines), quadratic classifiers, Kernel estimation (for e.g., k-nearest neighbor), Boosting, Decision trees (for e.g., Random forests), Neural networks, Bayesian networks, Hidden Markov models, or Learning vector quantization.

Once the multipotency index is obtained, it can be applied to methylation data sets obtained from any subject T cell (e.g., CD8 T cell) of interest to assess the differentiation stage of the subject T cell. Specifically, DNA methylation in subject T cells (e.g., CD8 T cells) can be determined at the one or more genomic sites of interest (e.g., one or more CpG sites, such as the CpG sites described in Table 1) and can be compared to the multipotency index to generate a multipotency score that is indicative of the differentiation potential of the subject T cell. In one embodiment, the multipotency score is a weighted dot product of the subject T cell (e.g., CD8 T cell) methylation status and a multipotency index, in which each methylation site is assigned a weighted score. In some embodiments, the multipotency score can be a normalized multipotency score. For example, the multipotency score can be normalized to a range of 0 to 1, where data sets with multipotency scores closer to 1 are more similar to naïve cells.

In some embodiments, the multipotency index is based on the DNA methylation status of naïve T cells, stem memory T cells (Tscm), self-reactive T cells (e.g., beta cell-specific T cells), central memory T cells (Tcm), and/or effector memory T cells (Tem). Such methylation data can be used in training sets in a machine learning approach to generate the multipotency index described herein. For example, the multipotency index may be based on DNA methylation profiles generated from two or more different types of T cells (e.g., CD8 T cells) selected from naïve T cells, stem memory T cells (Tscm), self-reactive T cells (e.g., beta cell-specific T cells), central memory T cells (Tcm), or effector memory T cells (Tem). In certain instances, the multipotency index is based on the DNA methylation status of one or more populations of naïve T cells (e.g., naïve CD8 T cells) and one or more populations of T cells (e.g., CD8 T cells) that have undergone further differentiation relative to naïve T cells (e.g., effector memory T cells or cells similar thereto, including antigen-specific (e.g., HIV) T cells).

In particular embodiments, the multipotency index is based on the DNA methylation status of two or more different types of CD8 T cells selected from naïve CD8 T cells, stem memory CD8 T cells (Tscm), self-reactive CD8 T cells (e.g., beta cell-specific CD8 T cells), central memory CD8 T cells (Tcm), and/or effector memory CD8 T cells (Tem). As used herein, the term “CD8+ T cell” or “CD8+ T lymphocyte” refers to a T cell that expresses CD8 on the surface thereof. CD8+ T cells include naive CD8+ T cells, cytotoxic T lymphocytes (CTLs), stem memory CD8+ T cell (CD8+ Tsmc), central memory CD8+ T cells (CD8+ TCM), effector memory CD8+ T cells (CD8+ TEM), effector CD8+ T cells (TE), or any combination thereof.

As used herein, the term “naive CD8+ T cell” refers to a non-antigen experienced CD8+ T cell that expresses CD62L and CD45RA, and does not express or has decreased expression of CD45RO− as compared to central memory CD4+ cells. In some embodiments, naive CD8+ T cells are characterized by the expression of phenotypic markers of naive T cells including CD62L, CCR7, CD28, CD3, CD127, and CD45RA.

As used herein, the term “cytotoxic T cell,” also known as Tc, cytotoxic T lymphocyte, CTL, killer T cell, or cytolytic T cell, refers to an activated, as opposed to naive, T lymphocyte that expresses CD8 on its surface. Naive CD8+ T cells become CTLs following activation by interacting with a WIC class I-restricted peptide antigen complex and co-stimulation via CD28. CD8+ CTLs include both effector CD8+ T cells and memory CD8+ T cells.

As used herein, the term “effector CD8+ T cells” (CD8+ TE) refer to antigen experienced CTLs that do not express or have decreased expression of CD62L, CCR7, CD28, and are positive for granzyme B and perforin as compared to central memory CD8+ T cells. Effector CD8+ T cells possess cytotoxic activity towards cells expressing the target antigen and are short lived as compared to CD8+ TM cells.

As used herein, the term “memory CD8+ T cells” (CD8+ TM) are antigen experienced CD8+ T cells that provide long lasting immunity. Memory CD8+ T cells are long lived, inactive CD8+ T cells that are able to rapidly acquire effector functions upon antigen re-challenge. Memory CD8+ T cells include stem memory CD8+ T cells (Tscm), central memory (TCM) CD8+ T cells, and effector memory CD8+ T cells (TE).

As used herein, “central memory CD8+ T cell” (CD8+ TCM) refers to an antigen experienced CTL that expresses CD62L and CD45RO on the surface thereof, and does not express or has decreased expression of CD45RA as compared to naive CD8+ T cells. Central memory CD8+ T cells have a longer lifespan than CD8+ TE and CD8+ TEM cells and can differentiate into effector memory CD8+ T cells following antigenic challenge. In some embodiments, central memory CD8+ T cells are positive for expression CD62L, CCR7, CD28, CD127, CD45RO, and CD95, and have decreased expression of CD54RA as compared to naive CD8+ T cells.

As used herein, “effector memory CD8+ T cell” (CD8+ TEM) refers to an antigen experienced CTL that does not express or has decreased expression of CD62L on the surface thereof as compared to central memory cells, and does not express or has decreased expression of CD45RA as compared to naive CD8+ T cells. Effector memory CD8+ T cells are terminally differentiated and acquire effector function immediately after re-stimulation by the same antigen. In some embodiments, effector memory CD8+ T cells are negative for expression CD62L, CCR7, CD28, CD45RA, and are positive for CD127 as compared to naive cells or central memory cells.

As used herein, “stem memory CD8+ T cell” (TSCM) refers to an antigen experienced CTL that expresses CD45RA, CD62L, CD95, and CD122. TSCM cells possess memory T cell capability of rapid acquisition of effector function following antigen re-challenge, but have enhanced stem cell-like qualities compared to TCM cells. TSCM cells can generate central memory, effector memory, and effector T cell subsets.

The terms “methylation status” or “methylation level” refer to the presence, absence, and/or quantity of methylation at a particular nucleotide, or nucleotides within a portion of DNA. The methylation status of a particular DNA sequence (e.g., a DNA biomarker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value” or “methylation level.” A methylation value or level can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, represents the methylation status and can thus be used as a quantitative indicator of methylation status. This is of particular use when it is desirable to compare the methylation status of a sequence in a sample to a threshold value. A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC).

The terms “measuring” and “determining” are used interchangeably throughout, and refer to methods which include obtaining a subject sample and/or detecting the methylation status or level of a biomarker(s) in a sample. In one embodiment, the terms refer to obtaining a subject sample and detecting the methylation status or level of one or more biomarkers in the sample. In another embodiment, the terms “measuring” and “determining” mean detecting the methylation status or level of one or more biomarkers in a subject sample. Measuring can be accomplished by methods known in the art and those further described herein including, but not limited to, quantitative polymerase chain reaction (PCR). The term “measuring” is also used interchangeably throughout with the term “detecting.”

The methylation status of certain genomic loci, or combinations thereof, can be used to identify the differentiation stage of the corresponding T cell (e.g., CD8 T cell). Based on a machine learning-based analysis (e.g., supervised analysis) of T cells (e.g., CD8 T cells), particular methylation sites (e.g., one or more CpG sites, such as the CpG sites described in Table 1) that are predictive of T cell differentiation potential can be identified. For example, the supervised analysis can be used to identify one or more CpG sites (e.g., one or more CpG sites, such as the CpG sites described in Table 1) that are hypomethylated in naïve T cells (e.g., naïve CD8 T cells) compared to antigen-specific T cells (e.g., antigen-specific CD8 T cells, such as HIV-specific CD8 T cells), thereby indicating that the methylation sites of interest are differentially methylated in different subsets of T cells. Methylation at the genomic sites of interest (e.g., one or more CpG sites, such as the CpG sites described in Table 1) can then be assessed via statistical methods to calculate a weighted score for each site that indicates the degree to which the particular site is predictive of differentiation status.

In specific CpG sites or “CpG islands” in the genome of a T cell (e.g., CD8 T cell) can be used to predict the stage of differentiation, and the corresponding T cell differentiation potential, of the T cell. The term “CpG islands” refers to a region of genomic DNA which shows higher frequency of 5′-CG-3′ (CpG) dinucleotides than other regions (i.e., control regions) of genomic DNA. CpG sites can also be found in a region with a low frequency of CpG sites such that the sites do not exist in a CpG island. Methylation of DNA at CpG dinucleotides, in particular, the addition of a methyl group to position 5 of the cytosine ring at CpG dinucleotides, is one of the epigenetic modifications in mammalian cells. CpG islands often harbor the promoters of genes and play a pivotal role in the control of gene expression. In normal tissues CpG islands are usually unmethylated, but a subset of islands becomes methylated during the development of a disease or condition. In certain embodiments, the methylation status of a CD8 T cell is based on the methylation status of one or more CpG sites in the T cell genome (e.g., one, two or more, three or more, five or more, ten or more, 25 or more, 50 or more, 75 or more, 100 or more, 125 or more, 150 or more, 175 or more, 200 or more, 225 or more, 250 or more, 300 or more, 350 or more, 400 or more, or 500 or more CpG sites). In particular embodiments, the methylation status of a CD8 T cell is based on the methylation status of one or more CpG sites in Table 1 (e.g., one, two or more, three or more, five or more, ten or more, 25 or more, 50 or more, 75 or more, 100 or more, 125 or more, 150 or more, 175 or more, 200 or more, 225 or more, or all 245 CpG sites in Table 1). In yet further embodiments, the methylation status of a CD8 T cell is based on the methylation status of wherein the measurement step comprises measuring the methylation status of at least 5%, at least 10%, at least 20%, at least 30%, at least 40%, at least 50%, at least 60%, at least 70%, at least 80%, or at least 90% of the 245 CpG sites in Table 1.

Accordingly, in some embodiments, the methods herein can be useful to identify the multipotency of subject T cells (e.g., CD8 T cells) isolated from a patient. Such methods are useful in instances where monitoring the stage of differentiation of T cells (e.g., CD8 T cells) in a subject can help stratify patients into those who may benefit from further monitoring or therapeutics. For example, in the case of autoimmune diseases, such as type-1 diabetes, auto-reactive T cells (e.g., auto-reactive CD8 T cells) have a high differentiation potential, as described in Example 1 herein. The methods herein are therefore useful for identifying patients who have potential autoimmune reactivity (e.g., type 1 diabetes) based on the multipotency score of T cells (e.g., CD8 T cells) isolated from the patient. Individuals having T cells (e.g., CD8 T cells) displaying an increased multipotency score relative to a control (e.g., T cells from a healthy individual or a pre-defined threshold level) may have a high level of auto-reactive T cells (e.g., auto-reactive CD8 T cells) and may therefore require further monitoring or therapeutics. In contrast, individuals having T cells (e.g., CD8 T cells) displaying a decreased multipotency score relative to a control (e.g., T cells from a healthy individual, T cells isolated from the individual at a previous time point, or a pre-defined threshold level) may have a reduced level of auto-reactive T cells (e.g., auto-reactive CD8 T cells), thereby indicating induction of T cell tolerance. Accordingly, individuals undergoing therapy for an autoimmune disease (e.g., type 1 diabetes) may be monitored for therapeutic efficacy based on changes in the multipotency score of T cells (e.g., CD8 T cells) assessed by the methods described herein.

As used herein, the term “autoimmune disease” refers to any disease in which the body produces an immunogenic (ie, immune system) response to some constituent of its own tissue. In other words, the immune system loses its ability to recognize some tissue or system within the body as “self” and targets and attacks it as if it were foreign. Autoimmune diseases can be classified into those in which predominantly one organ is affected (eg, hemolytic anemia and anti-immune thyroiditis), and those in which the autoimmune disease process is diffused through many tissues (eg, systemic lupus erythematosus). Examples of autoimmune diseases include, but are not limited to, rheumatoid arthritis, multiple sclerosis, lupus erythematosis, myasthenia gravis, scleroderma, Crohn's disease, ulcerative colitis, Hashimoto's disease, Graves' disease, Sjogren's syndrome, polyendocrine failure, vitiligo, peripheral neuropathy, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barr syndrome, hemochromatosis, Henoch-Schonlein purpura, idiopathic IgA nephropathy, type 1 diabetes, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, inflammatory bowel disease, osteoarthritis and thyroiditis.

In some embodiments, the methods herein can be useful to identify and isolate T cells (e.g., CD8 T cells) having enhanced differentiation potential and preserved effector capacity or T cell activity. For example, CD8 T cells undergo activation by interaction of the T-cell receptor (TCR) on the CD8 T cell with antigen bound to MHC-I on antigen presenting cells. Once activated the T cell undergoes clonal expansion to increase the number of cells specific for the target antigen. When exposed to infected or dysfunctional somatic cells having the specific antigen for which the TCR is specific, the activated CD8 T cells release cytokines and cytotoxins to eliminate the infected or dysfunctional cell. The release of specific cytokines and cytotoxins by CD8 T cells in response to an antigen is referred to herein as “effector functions”. Likewise, the term “effector potential” refers to the ability of CD8 T cells to activate effector functions upon TCR engagement. The term “T cell activity” refers to any of the following: cytokine production (e.g., IFNγ and IL-2) upon TCR engagement; expression of cytotoxic molecules (e.g., granzyme B and perforin) upon TCR engagement; rapid cell division upon TCR engagement; cytolysis of antigen presenting cells; IL-7 and IL-15 mediated homeostatic proliferation; and in vivo trafficking to lymphoid tissues or sites of antigen presentation. Moreover, “T cell activity” can refer to the persistence of immunological memory in the absence of antigen.

In particular embodiments, the T cell is a T cell (e.g., CD8 T cell) having a modified T-cell receptor, such as a CAR T cell. As used herein, a “chimeric antigen receptor” or “CAR” refers to an engineered receptor that grafts specificity for an antigen onto an immune effector cell (e.g., a human T cell). A chimeric antigen receptor typically comprises an extracellular ligand-binding domain or moiety and an intracellular domain that comprises one or more stimulatory domains. In some embodiments, the extracellular ligand-binding domain or moiety can be in the form of single-chain variable fragments (scFvs) derived from a monoclonal antibody, which provide specificity for a particular epitope or antigen (e.g., an epitope or antigen preferentially present on the surface of a cancer cell or other disease-causing cell or particle). The extracellular ligand-binding domain can be specific for any antigen or epitope of interest.

T-cell adoptive immunotherapy is a promising approach for cancer treatment. This strategy utilizes isolated human T cells (e.g., CD8 T cells) that have been genetically-modified to enhance their specificity for a specific tumor associated antigen. Genetic modification may involve the expression of a chimeric antigen receptor or an exogenous T cell receptor to graft antigen specificity onto the T cell. By contrast to exogenous T cell receptors, chimeric antigen receptors derive their specificity from the variable domains of a monoclonal antibody. Thus, CAR T cells induce tumor immunoreactivity in a major histocompatibility complex non-restricted manner. To date, T cell adoptive immunotherapy has been utilized as a clinical therapy for a number of cancers, including B cell malignancies (e.g., acute lymphoblastic leukemia (ALL), B cell non-Hodgkin lymphoma (NHL), and chronic lymphocytic leukemia), multiple myeloma, neuroblastoma, glioblastoma, advanced gliomas, ovarian cancer, mesothelioma, melanoma, and pancreatic cancer, among others. In some embodiments, CAR T cells having modulated methylation profiles are administered along with ICB therapy.

In specific embodiments, CAR-CD8 T cells may be adoptively transferred into a patient. Adoptive transfer T cell therapy of methylase-deficient CD8 T cells may also be used in combination with immune checkpoint inhibitors such as antibodies to PD-1/PD-L1 and/or CD80/CTLA4 blockade, small molecule checkpoint inhibitors, interleukins, e.g., IL-2 (aldesleukin).

In some embodiments, T-cells (e.g., CD8 T cells) of the methods or compositions herein can be administered to a patient having a chronic infection, autoimmune disease, or cancer. In some embodiments, the chronic infection is a chronic viral infection. For example, T-cells can be administered using the methods disclosed herein in a subject infected with influenza A virus including subtype H1N1, influenza B virus, influenza C virus, rotavirus A, rotavirus B, rotavirus C, rotavirus D, rotavirus E, SARS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E, or human immunodeficiency virus (HIV). In particular embodiment, the chronic viral infection is HIV, HCV, and/or herpes virus.

In some embodiments, T cells (e.g., CD8 T cells) can be administered using the methods disclosed herein to a subject having autoimmune disease. Examples of autoimmune diseases include, but are not limited to, rheumatoid arthritis, multiple sclerosis, lupus erythematosis, myasthenia gravis, scleroderma, Crohn's disease, ulcerative colitis, Hashimoto's disease, Graves' disease, Sjogren's syndrome, polyendocrine failure, vitiligo, peripheral neuropathy, autoimmune polyglandular syndrome type I, acute glomerulonephritis, Addison's disease, adult-onset idiopathic hypoparathyroidism (AOIH), alopecia totalis, amyotrophic lateral sclerosis, ankylosing spondylitis, autoimmune aplastic anemia, autoimmune hemolytic anemia, Behcet's disease, Celiac disease, chronic active hepatitis, CREST syndrome, dermatomyositis, dilated cardiomyopathy, eosinophilia-myalgia syndrome, epidermolisis bullosa acquisita (EBA), giant cell arteritis, Goodpasture's syndrome, Guillain-Barr syndrome, hemochromatosis, Henoch-Schonlein purpura, idiopathic IgA nephropathy, type 1 diabetes, juvenile rheumatoid arthritis, Lambert-Eaton syndrome, linear IgA dermatosis, myocarditis, narcolepsy, necrotizing vasculitis, neonatal lupus syndrome (NLE), nephrotic syndrome, pemphigoid, pemphigus, polymyositis, primary sclerosing cholangitis, psoriasis, rapidly-progressive glomerulonephritis (RPGN), Reiter's syndrome, stiff-man syndrome, inflammatory bowel disease, osteoarthritis and thyroiditis.

As used herein a “proliferative disease” or “cancer” includes, a disease, condition, trait, genotype or phenotype characterized by unregulated cell growth or replication as is known in the art; including leukemias, for example, acute myelogenous leukemia (AML), chronic myelogenous leukemia (CIVIL), acute lymphocytic leukemia (ALL), and chronic lymphocytic leukemia, AIDS related cancers such as Kaposi's sarcoma; breast cancers; bone cancers such as osteosarcoma, chondrosarcomas, Ewing's sarcoma, fibrosarcomas, giant cell tumors, adamantinomas, and chordomas; brain cancers such as meningiomas, glioblastomas, lower-grade astrocytomas, oligodendrocytomas, pituitary tumors, schwannomas, and metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of disease related gene expression in a cell or tissue, alone or in combination with other therapies.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

In some embodiments, the T cells (e.g., CD8 T cells) described herein are modified to express a chimeric antigen receptor (CAR) specific to a tumor associated antigen or neoantigen. In certain embodiments, the tumor associated antigen is selected from CD5, CD19, CD20, CD30, CD33, CD47, CD52, CD152(CTLA-4), CD274(PD-L1), CD340(ErbB-2), GD2, TPBG, CA-125, CEA, MAGEA1, MAGEA3, MART1, GP100, MUC1, WT1, TAG-72, HPVE6, HPVE7, BING-4, SAP-1, immature laminin receptor, vascular endothelial growth factor (VEGFA) or epidermal growth factor receptor (ErbB-1). In certain embodiments, the tumor associated antigen is selected from CD20, CD20, CD30, CD33, CD52, EpCAM, epithelial cells adhesion molecule, gpA33, glycoprotein A33, Mucins, TAG-72, tumor-associated glycoprotein 72, Folate-binding protein, VEGF, vascular endothelial growth factor, integrin αVβ3, integrin α5β1, FAP, fibroblast activation protein, CEA, carcinoembryonic antigen, tenascin, Ley, Lewis Y antigen, CAIX, carbonic anhydrase IX, epidermal growth factor receptor (EGFR; also known as ERBB1), ERBB2 (also known as HER2), ERBB3, MET (also known as HGFR), insulin-like growth factor 1 receptor (IGF1R), ephrin receptor A3 (EPHA3), tumor necrosis factor (TNF)-related apoptosis-inducing ligand receptor 1 (TRAILR1; also known as TNFRSF10A), TRAILR2 (also known as TNFRSF10B) and receptor activator of nuclear factor-KB ligand (RANKL; also known as TNFSF11) and fragments thereof.

In certain embodiments, the T-cells (e.g., CD8 T cells) specific to a tumor antigen can be removed from a tumor sample (TILs) or filtered from blood. Subsequent activation and culturing is performed outside the body (ex vivo) and then they are transfused into the patient. Activation may be accomplished by exposing the T cells (e.g., CD8 T cells) to tumor antigens. In certain embodiments, the tumor antigen is a low-binding affinity TCR epitope.

### III. Methods for Isolating a Subset of T Cells and Methods of Use

Methods and compositions are provided herein for selecting and isolating a population of T cells (e.g., CD8 T cells) that have a desired multipotency or stage of differentiation based on the methylation status of a specific locus or combination of loci or the methylation profile of a genomic region or complete genome of a T cell (e.g., CD8 T cell). Selection and isolation of a subset of T cells (e.g., CD8 T cells) with a desired differentiation potential can be performed by measuring the methylation status of a combination of loci or the methylation profile of a genomic region or complete genome of a sample of T cells (e.g., CD8 T cells) in order to predict the T cell differentiation potential of the population from which the sample was taken.

Accordingly, in one aspect, provided herein is a method of isolating a population of T cells (e.g., CD8 T cells) with a desired differentiation potential. This method involves dividing a starting population of T cells (e.g., CD8 T cells) into three or more subpopulations (e.g., 3 or more, 4 or more, 5 or more, 6 or more, 7 or more, 8 or more, 9 or more, or 10 or more subpopulations), determining the multipotency score for each subpopulation, and subsequently identifying and combining the subpopulations with the desired multipotency score to produce a final population of T cells. In certain instances, the method involves the steps of (a) measuring the methylation status of the T cells (e.g., CD8 T cells) in each subpopulation; (b) establishing a multipotency score based on a comparison of the methylation status to a T cell multipotency index; (c) identifying subpopulations of T cells having the desired differentiation potential (e.g., increased differentiation potential) based on the multipotency score; and (d) combining at least two identified subpopulations of T cells having the desired differentiation potential (e.g., increased differentiation potential) into a final population of T cells, wherein at least one subpopulation of the starting population of T cells is not combined into the final population of T cells. In some embodiments, this method may be used to isolate a population of T cells (e.g., CD8 T cells) at a specific differentiation stage (e.g., naïve T cells, stem memory T cells, central memory T cells, effector memory T cells, or effector memory-like T cells). In certain embodiments, the methods provided herein may be used to isolate a population of T cells (e.g., CD8 T cells) with improved or increased differentiation potential. For example, the final population of T cells (e.g., CD8 T cells) may have an increased multipotency score, and a corresponding increase in differentiation potential, relative to a control or a natural population of T cells from the same origin. In other embodiments, the multipotency score of the final population of T cells (e.g., CD8 T cells) may be increased relative to a pre-defined threshold.

The methylation status of any individual locus or a group of loci in the genome of a T cell (e.g., CD8 T cell) can be measured by any means known in the art or described herein. For example, methylation can be determined by methylation-specific PCR, whole genome bisulfite sequencing, locus specific bisulfite sequencing, Ingenuity Pathway Analysis (IPA), the HELP assay and other methods using methylation-sensitive restriction endonucleases, ChIP-on-chip assays, restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methyl CpG binding proteins, mass spectrometry, HPLC, and reduced representation bisulfate sequencing. In some embodiments methylation is detected at specific sites of DNA methylation using pyrosequencing after bisulfite treatment and optionally after amplification of the methylation sites. Pyrosequencing technology is a method of sequencing-by-synthesis in real time. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray.

In specific embodiments, detection of DNA methylation can be performed by first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. For example, isolated DNA can be treated with sodium bisulfite (NaHSO3) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore, after PCR or sequencing, the resultant product contains cytosine only at the position where 5-methylcytosine occurs in the starting template DNA. This makes the discrimination between unmethylated and methylated cytosine possible.

The methylation status of CpG sites in test and controls samples may be compared by calculating the proportion of discordant reads, calculating variance, or calculating information entropy identifying differentially methylated regions, by quantifying methylation difference, or by gene-set analysis (i.e., pathway analysis), preferably by calculating the proportion of discordant reads, calculating variance, or calculating information entropy. Optionally, information entropy is calculated by adapting Shannon entropy. In some embodiments, gene-set analysis is performed by tools such as DAVID, GoSeq or GSEA. In some embodiments, a proportion of discordant reads (PDR) is calculated. Optionally, each region of neighboring CpG sites (e.g., within a sequencing read) is assigned a consistent status or an inconsistent status before calculating the proportion of discordant reads, variance, epipolymorphism or information entropy. There may be multiple inconsistent statuses, each representing a distinct methylation pattern or class of similar methylation patterns.

The CpG site identified for methylation analysis can be in a genomic feature selected from a CpG island, a CpG shore, a CpG shelf, a promoter, an enhancer, an exon, an intron, a gene body, a stem cell associated region, a short interspersed element (SINE), a long interspersed element (LINE), and a long terminal repeat (LTR). In specific embodiments, the CpG site is in a CpG island, a transcription factor, or a promoter within a given genomic locus.

In some embodiments, the stage of differentiation or multipotency of a T cell (e.g., CD8 T cell) can be predicted based on the methylation status of a combination of genomic loci (e.g., one or more CpG sites, such as the CpG sites described in Table 1). In certain embodiments, the methylation status of a T cell (e.g., CD8 T cell) is based on the methylation status of one or more CpG sites in the T cell genome (e.g., one, two or more, three or more, five or more, ten or more, 25 or more, 50 or more, 75 or more, 100 or more, 125 or more, 150 or more, 175 or more, 200 or more, 225 or more, 250 or more, 300 or more, 350 or more, 400 or more, or 500 or more CpG sites). In particular embodiments, the methylation status of a T cell (e.g., CD8 T cell) is based on the methylation status of one or more CpG sites in Table 1 (e.g., one, two or more, three or more, five or more, ten or more, 25 or more, 50 or more, 75 or more, 100 or more, 125 or more, 150 or more, 175 or more, 200 or more, 225 or more, or all 245 CpG sites in Table 1). In yet further embodiments, the methylation status of a T cell (e.g., CD8 T cell) is based on the methylation status of wherein the measurement step comprises measuring the methylation status of at least 5%, at least 10%, at least 20%, at least 30%, at least 40%, at least 50%, at least 60%, at least 70%, at least 80%, or at least 90% of the 245 CpG sites in Table 1. Accordingly, an increased multipotency score based on particular methylation sites relative to the corresponding multipotency score for the same sites of an appropriate control indicates increased T-cell differentiation potential compared to the control. Likewise, a decreased multipotency score for particular methylation sites relative to the corresponding multipotency score for the same sites of an appropriate control indicates decreased T-cell differentiation potential compared to the control.

An appropriate control can be readily selected by one skilled in the art. For example, an appropriate control may be a multipotency score from T cells (e.g., CD8 T cells) from a starting sample or T cells of a known differentiation stage (naïve T cells, stem memory T cells, self-reactive T cells, central memory T cells, effector memory T cells, or antigen-specific T cells). For example, in certain instances, the control may be a natural population of T cells (e.g., CD8 T cells) from the same origin as a T cell population isolated by the methods provided herein. In some instances, in methods that involve monitoring a patient for changes in T cell (e.g., CD8 T cell) multipotency (e.g., tolerance induction), the control may be the multipotency score for T cells isolated from the same patient at a previous time point, or from T cells isolated from a different individual (e.g., a healthy individual or a patient having the disease). In particular embodiments, a control can be the multipotency score of T cells (e.g., CD8 T cells) obtained from a sample obtained from a normal individual (e.g., normal or control tissue, or normal or control body fluid, stool, blood, serum, amniotic fluid), most importantly in healthy stool, blood, serum, amniotic fluid or other body fluid.

In some instances, the multipotency score for a given sample is compared to a threshold multipotency score. For example, in instances where the multipotency score is normalized to a range of 0 to 1, T cells (e.g., CD8 T cells) with multipotency scores closer to 1 are more similar to naïve cells and T cells (e.g., CD8 T cells) with multipotency scores closer to 0 are more similar to antigen-specific T cells (e.g., effector memory T cells). For example, a multipotency score above a threshold of 0.1 0.2, 0.3, 0.4, 0.5, 0.6, 0.7, 0.8, or 0.9 may be indicative of T cells (e.g., CD8 T cells) having a high degree of multipotency (e.g., naïve T cells, TSCM stem cells, or self-reactive stem cells). In certain instances, a multipotency score above 0.3 is indicative of a T cells (e.g., CD8 T cells) having a high degree of multipotency. In certain instances, a multipotency score above 0.4 is indicative of a T cells (e.g., CD8 T cells) having a high degree of multipotency. In certain instances, a multipotency score above 0.5 is indicative of a T cells (e.g., CD8 T cells) having a high degree of multipotency. Alternatively, a multipotency score below a threshold of 0.1, 0.2, 0.3, 0.4, 0.5, 0.6, 0.7, 0.8, 0.9, or 1 may be indicative of a T cells (e.g., CD8 T cells) having a low degree of multipotency (e.g., TEM, TCM, or TEM-like antigen-specific cells). In certain instances, a multipotency score below 0.3 is indicative of a T cells (e.g., CD8 T cells) having a low degree of multipotency. In certain instances, a multipotency score below 0.2 is indicative of a T cells (e.g., CD8 T cells) having a low degree of multipotency. In certain instances, a multipotency score below 0.1 is indicative of a T cells (e.g., CD8 T cells) having a low degree of multipotency

Further, the methods herein can further include testing the methylation status of the loci of effector cytokines, transcription factors, or regulators of cellular proliferation to predict T-cell (e.g., CD8 T cell) differentiation potential. For example, the methylation status of genes, promoters, and/or transcription factors of DNTM3a, TOX, BATF, IFNγ, granzyme K (GzmK), granzyme B (GzmB), Prf1, T-bet, Tcf7, Myc, T-bet, eomesodermin (Eomes), Foxp1, CCR7, and/or CD62L can be used for prediction of T-cell (e.g., CD8 T cell) differentiation potential, as described elsewhere herein. In certain instances, the methylation status of genes, promoters, and/or transcription factors of DNTM3a, TOX, and BATF can be used to assist in identifying the differentiation stage or potential for T cells. For example, T cells with increased multipotency relative to a control may have increased methylation of the TOX locus relative to the control, increased methylation of BATF relative to the control, and/or decreased methylation or unmethylated DNTM3a relative to the control. The methylation status of an individual marker locus (e.g., DNTM3a, TOX, BATF) can be measured at any location within the marker locus. The marker locus (e.g., DNTM3a, TOX, BATF) can refer to a CpG site within the marker locus. As used herein a marker locus includes, but is not limited to, the genomic region beginning 2 kb upstream of the transcription start site and ending 2 kb downstream of the stop codon for each marker locus. The marker locus can include the region beginning 1 kb upstream of the transcription start site and ending 1 kb downstream of the stop codon, beginning 500 bp upstream of the transcription start site and ending 500 bp downstream of the stop codon, beginning 250 bp upstream of the transcription start site and ending 250 bp downstream of the stop codon, beginning 100 bp upstream of the transcription start site and ending 100 bp downstream of the stop codon, beginning 50 bp upstream of the transcription start site and ending 50 bp downstream of the stop codon, or beginning 10 bp upstream of the transcription start site and ending 10 bp downstream of the stop codon of the marker gene. In specific embodiments, the methylation status of an individual marker gene can be measured at a CpG site within the genomic locus.

Populations of T cells (e.g., CD8 T cells) having a desired activity can be selected based on the methylation status of an individual locus or a combination of loci of a sample of T cells taken from the population. In some embodiments, T cell (e.g., CD8 T cell) populations are selected based on a multipotency score determined from measurement of the methylation status of any locus listed herein (e.g., a CpG site listed in Table 1). In specific embodiments, selected T-cell (e.g., CD8 T cell) populations may comprise at least 30%, 40%, 50%, 55%, 60%, 65%, 70%, 75%, 80%, 90%, 95%, or more CD8 T cells having a multipotency score higher than a control sample or threshold multipotency score. In other embodiments, selected T-cell (e.g., CD8 T cell) populations may comprise at least 30%, 40%, 50%, 55%, 60%, 65%, 70%, 75%, 80%, 90%, 95%, or more T cells (e.g., CD8 T cells) having a multipotency score lower than a control sample or threshold multipotency score.

In certain embodiments, the present invention provides for a pharmaceutical composition comprising a T cell (e.g., CD8 T cell) selected by the method disclosed herein, or comprising a population of CD8 T cells (e.g., CD8 T cells) comprising at least 30%, 40%, 50%, 55%, 60%, 65%, 70%, 75%, 80%, 90%, 95%, or more T cells (e.g., CD8 T cells) having a multipotency score indicative of increased differentiation potential (e.g., relative to a control or threshold score), as disclosed herein. The T cell (e.g., CD8 T cell), or T cell (e.g., CD8 T cell) population can be suitably formulated and introduced into a subject or the environment of the cell by any means recognized for such delivery. In some embodiments, the pharmaceutical composition comprises a CAR T cell produced from a T cell (e.g., CD8 T cells) selected based on the identification and isolation of T cells having a multipotency score indicative of increased differentiation potential (e.g., relative to a control or threshold score).

Such pharmaceutical compositions typically include the agent (e.g., T cell, such as a CD8 T cell) and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. In some embodiment a synthetic carrier is used wherein the carrier does not exist in nature. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The pharmaceutical compositions can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies with the T cells disclosed herein can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an T cell or demethylating agent (including, e.g., a protein, polypeptide, or antibody) can include a single treatment or, preferably, can include a series of treatments.

The pharmaceutical compositions can be included in a kit, container, pack, or dispenser together with instructions for administration.

The present invention provides for both prophylactic and therapeutic methods of treating a subject at risk of (or susceptible to) a chronic disease or infection. “Treatment”, or “treating” as used herein, is defined as the application or administration of a therapeutic agent (e.g., a selected T cell) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., a selected T cell). Subjects at risk for the disease can be identified by, for example, one or a combination of diagnostic or prognostic assays as known in the art. Administration of a prophylactic agent can occur prior to the detection of, e.g., cancer in a subject, or the manifestation of symptoms characteristic of the disease or disorder, such that the disease or disorder is prevented or, alternatively, delayed in its progression.

Another aspect of the invention pertains to methods of treating subjects therapeutically, i.e., altering the onset of symptoms of the disease or disorder. These methods can be performed in vitro (e.g., by culturing the cell with the agent(s)) or, alternatively, in vivo (e.g., by administering the agent(s) to a subject). With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment according to that individual's drug response genotype, methylation profile, expression profile, biomarkers, etc. Pharmacogenomics allows a clinician or physician to target prophylactic or therapeutic treatments to patients who will most benefit from the treatment and to avoid treatment of patients who will experience toxic drug-related side effects.

Therapeutic agents (e.g., a T cell, such as a CD8 T cell) can be tested in a selected animal model. For example, a pharmaceutical composition (e.g., comprising an isolated T cell, such as a CD8 T cell) having a multipotency score indicative of increased differentiation potential (e.g., relative to a control) as described herein can be used in an animal model to determine the efficacy, toxicity, or side effects of treatment with said agent. Alternatively, an agent (e.g., a T cell, such as a CD8 T cell) can be used in an animal model to determine the mechanism of action of such an agent. Accordingly, methods are provided herein for the treatment or prevention of a chronic infection or cancer by administering a T cell (e.g., CD8 T cell) or CAR T cell (e.g., CAR CD8 T cell) having a desired T cell differentiation potential selected based on the methods described herein.

In another aspect, provided herein is a kit comprising reagents for detecting the methylation status of one or more CpG sites in a T cell (e.g., CD8 T cell). For example, the one or more CpG sites can include one, two or more, three or more, five or more, ten or more, 25 or more, 50 or more, 75 or more, 100 or more, 125 or more, 150 or more, 175 or more, 200 or more, 225 or more, 250 or more, 300 or more, 350 or more, 400 or more, or 500 or more CpG sites. In certain instances, the kit can include reagents for detecting the methylation status of one or more CpG sites selected from Table 1 (e.g., one, two or more, three or more, five or more, ten or more, 25 or more, 50 or more, 75 or more, 100 or more, 125 or more, 150 or more, 175 or more, 200 or more, 225 or more, or all 245 CpG sites in Table 1). In yet further embodiments, the kit can include reagents for detecting the methylation status of at least 5%, at least 10%, at least 20%, at least 30%, at least 40%, at least 50%, at least 60%, at least 70%, at least 80%, or at least 90%, or 100% of the 245 CpG sites in Table 1. The kit can further include instructions for accessing, utilizing, and/or generating a T cell multipotency index. The instructions can be in the form, for example, of a package insert. Alternatively, instructions may be accessible electronically (e.g., accessible via a website or a computer-readable medium (e.g., flash memory or other memory technology such as a CD-ROM or DVD).

### Embodiments

1. A method of identifying the stage of differentiation of subject T cells, said method comprising:
   a) measuring the methylation status of the subject T cells;
   b) establishing a multipotency score for the subject T cells based on a comparison of the methylation status of the subject T cells to a T-cell multipotency index;
   c) identifying the stage of differentiation of the subject T cells based on the multipotency score.
2. The method of embodiment 1, wherein the stage of differentiation is naïve T cells, stem memory T cells, self-reactive T cells, central memory T cells, or effector memory T cells.
3. The method of any one of embodiments 1-3, wherein the measurement step comprises measuring the methylation status of one or more CpG site in the subject T cells.
4. The method of embodiment 3, wherein the one or more CpG sites comprise one or more of the CpG sites selected from Table 1.
5. The method of embodiment 4, wherein the one or more CpG sites comprise two or more of the CpG sites selected from Table 1.
6. The method of any one of embodiments 1-3, wherein the measurement step comprises measuring the methylation status of each of the 245 CpG sites in Table 1.
7. The method of any one of embodiments 1-6, further comprising generating a multipotency index by identifying CpG sites that are differentially methylated in at least two populations of T cells and assigning a weighted index score to each CpG site.
8. The method of embodiment 7, wherein the CpG sites are identified by supervised analysis of training data sets comprising genome-wide methylation profiles of the least two populations of T cells, wherein the at least two populations of T cells are at different stages of differentiation.
9. The method of embodiment 8, where the T cells are selected from naïve T cells, stem memory T cells, central memory T cells, effector memory T cells, or effector memory-like T cells.
10. The method of embodiment 9, wherein the effector memory-like T cells are HIV-specific T cells.
11. The method of embodiment 10, wherein the at least two populations of T cells comprise one or more populations of naïve T cells and one or more populations of HIV-specific T cells.
12. The method of embodiment 9, wherein the at least two populations of T cells comprise one or more populations of naïve T cells and one or more populations of effector memory T cells.
13. The method of any one of embodiments 7-12, wherein one or more machine learning algorithm identifies the differentially methylated CpG sites and generates weighted index scores for each identified CpG site, thereby generating the multipotency index.
14. The method of embodiment 13, wherein the one or more machine learning algorithm is a one-class logistic regression algorithm.
15. The method of any one of embodiments 1-14, wherein the T cell multipotency index comprises one or more weighted index scores, wherein each weighted index score corresponds to a CpG site in a T cell genome.
16. The method of embodiment 15, wherein the step of establishing the multipotency score comprises determining the dot product between the methylation status of the subject T cells at the one or more CpG sites and the corresponding weighted index score for the CpG site.
17. The method of embodiment 15 or 16, wherein the weighted score corresponds to a CpG site selected from Table 1.
18. The method of embodiment 17, wherein the step of establishing the multipotency score comprises determining the dot product between the methylation status of the subject T cells at one or more CpG sites selected from Table 1 and the corresponding weighted index score in Table 1.
19. The method of embodiment 18, wherein the step of establishing the multipotency score comprises determining the dot product between the methylation status of the subject T cells at two or more CpG sites selected from Table 1 and the corresponding weighted index score in Table 1.
20. The method of embodiment 19, wherein the step of establishing the multipotency score comprises determining the dot product between the methylation status of the subject T cells at each CpG site selected from Table 1 and the corresponding weighted index score in Table 1.
21. The method of embodiment 1-20, wherein the multipotency score is a normalized multipotency score.
22. The method of embodiment 21, wherein the step of establishing the multipotency score comprises normalizing the multipotency score to a range of 0 to 1.
23. The method of embodiment 22, wherein a normalized multipotency score above 0.1, 0.2, 0.3, 0.4, 0.5, 0.6, 0.7, 0.8, or 0.9 indicates the subject T cells have a high differentiation potential and a score below 0.9, 0.8, 0.7, 0.6, 0.5, 0.4, 0.3, 0.2, or 0.1 indicates the subject T cells have a low differentiation potential.
24. The method of any one of embodiments 1-23, wherein a multipotency score higher than a control evaluated by the same method identifies the T cell as having increased differentiation potential relative to the control.
25. The method of any one of embodiments 1-23, wherein a multipotency score higher than a pre-established threshold score identifies the T cell as having increased differentiation potential.
26. The method of any one of embodiments 1-25, wherein the subject T cell is a CD8 T cell.
27. The method of embodiment 26, wherein the CD8 T cell is a human CD8 T cell.
28. The method of any one of embodiments 1-27, wherein the subject T cell is collected from a patient.
29. The method of embodiment 28, wherein the patient has cancer, an autoimmune disease, or a chronic infection.
30. The method of embodiment 29, wherein the autoimmune disease is type-1 diabetes.
31. The method of embodiment 29 or 30, wherein the method is effective to identify tolerance induction among the T cells collected from the patient.
32. The method of embodiment 31, wherein the T cells are self-reactive T cells.
33. The method of embodiment 32, wherein the T cells are beta cell-specific CD8 T cells.
34. The method of any one of embodiments 29-33, wherein the patient has been previously administered a therapeutic that induces T cell tolerance.
35. A method of isolating a population of T cells with improved differentiation potential, said method comprising:
   a) dividing a starting population of T cells into at least three subpopulations;
   b) measuring the methylation status of the T cells in each subpopulation;
   c) establishing a multipotency score based on a comparison of the methylation status to a T cell multipotency index;
   d) identifying subpopulations of T cells having increased differentiation potential based on the multipotency score; and
   e) combining at least two identified subpopulations of T cells having increased differentiation potential into a final population of T cells, wherein at least one subpopulation of the starting population of T cells is not combined into the final population of T cells.
36. The method of embodiment 35, wherein the differentiation potential of the final population of T cells is increased relative to the differentiation potential of a natural population of CD8 T cells from the same origin.
37. The method of embodiment 35, wherein the multipotency score of the final population of T cells is increased relative to a control.
38. The method of embodiment 35, wherein the multipotency score of the final population of T cells is increased relative to a pre-defined threshold.
39. The method of any one of embodiments 35-38, wherein the measurement step comprises measuring the methylation status of one or more CpG sites in the T cells.
40. The method of embodiment 39, wherein the one or more CpG sites comprise one or more of the CpG sites selected from Table 1.
41. The method of embodiment 40, wherein the one or more CpG sites comprise two or more of the CpG sites selected from Table 1.
42. The method of any one of embodiments 35-38, wherein the measurement step comprises measuring the methylation status of at least 5%, at least 10%, at least 20%, at least 30%, at least 40%, at least 50%, at least 60%, at least 70%, at least 80%, or at least 90% of the 245 CpG sites in Table 1.
43. The method of any one of embodiments 35-38, wherein the measurement step comprises measuring the methylation status of each of the 245 CpG sites in Table 1.
44. The method of any one of embodiments 35-43, further comprising generating a multipotency index by identifying CpG sites that are differentially methylated in the at least two populations of T cells and assigning a weighted index score to each CpG site.
45. The method of embodiment 44, wherein the CpG sites are identified by supervised analysis of training data sets comprising genome-wide methylation profiles of the least two populations of T cells, wherein the at least two populations of T cells are at different stages of differentiation.
46. The method of embodiment 45, where the T cells are selected from naïve T cells, stem memory T cells, central memory T cells, effector memory T cells, or effector memory-like T cells.
47. The method of embodiment 46, wherein the effector memory-like T cells are HIV-specific CD8 T cells.
48. The method of embodiment 45, wherein the at least two populations of T cells comprise one or more populations of naïve T cells and one or more populations of HIV-specific T cells.
49. The method of embodiment 45, wherein the at least two populations of T cells comprise one or more populations of naïve T cells and one or more populations of effector memory T cells.
50. The method of any one of embodiments 44-49, wherein one or more machine learning algorithms identifies the differentially methylated CpG sites and generates weighted index scores for each identified CpG site, thereby generating the multipotency index.
51. The method of embodiment 50, wherein the one or more machine learning algorithm is a one-class logistic regression algorithm.
52. The method of any one of embodiments 35-51, wherein the T cell multipotency index comprises one or more weighted index scores, wherein each weighted index score corresponds to a CpG site in a T cell genome.
53. The method of embodiment 52, wherein the step of establishing the multipotency score comprises determining the dot product between the methylation status of the subject T cells at the one or more CpG sites and the corresponding weighted index score for the CpG site.
54. The method of embodiment 52 or 53, wherein the weighted score corresponds to a CpG site selected from Table 1.
55. The method of embodiment 54, wherein the step of establishing the multipotency score comprises determining the dot product between the methylation status of the subject T cells at one or more CpG sites selected from Table 1 and the corresponding weighted index score in Table 1.
56. The method of embodiment 55, wherein the step of establishing the multipotency score comprises determining the dot product between the methylation status of the subject T cells at two or more CpG sites selected from Table 1 and the corresponding weighted index score in Table 1.
57. The method of embodiment 56, wherein the step of establishing the multipotency score comprises determining the dot product between the methylation status of the subject T cells at each CpG site selected from Table 1 and the corresponding weighted index score in Table 1.
58. The method of embodiment 35-57, wherein the multipotency score is a normalized multipotency score.
59. The method of embodiment 58, wherein the step of establishing the multipotency score comprises normalizing the multipotency score to a range of 0 to 1.
60. The method of embodiment 59, wherein a normalized multipotency score above 0.1, 0.2, 0.3, 0.4, 0.5, 0.6, 0.7, 0.8, or 0.9 indicates the subject T cells have high differentiation potential and a score below 0.9, 0.8, 0.7, 0.6, 0.5, 0.4, 0.3, 0.2, or 0.1 indicates the subject T cells have a low differentiation potential.
61. The method of any one of embodiments 35-60, wherein a multipotency score higher than a control a control identifies the T cells as having increased differentiation potential relative to the control.
62. The method of any one of embodiments 35-61, wherein a multipotency score higher than a pre-established threshold score identifies the T cell as having increased differentiation potential.
63. The method of any one of embodiments 35-62, wherein the subject T cell is a CD8 T cell.
64. The method of embodiment 63, wherein the CD8 T cell is a human CD8 T cell.
65. The method of any one of embodiments 35-64, wherein cells in the final population of T cells comprise a methylated TOX locus.
66. The method of any one of embodiments 35-65, wherein cells in the final population of T cells comprise an unmethylated DNMT3a locus.
67. The method of any one of embodiments 35-66, wherein cells in the final population of T cells comprise a methylated BATF locus.
68. A population of T cells isolated by the method of any one of embodiments 35-67, wherein the T cells have increased differentiation potential relative to a control.
69. A pharmaceutical composition comprising said population of CD8 T cells of embodiment 68.
70. The pharmaceutical composition of embodiment 69, wherein the pharmaceutical composition further comprises a pharmaceutically acceptable carrier.
71. A method of treating a chronic infection, an autoimmune disease, or a cancer in a subject, said method comprising administering to the subject the pharmaceutical composition of embodiment 69 or 70.
72. A method of monitoring T cell differentiation in a patient having an autoimmune disease, comprising:
   a) collecting a sample from the patient containing a population of T cells;
   b) measuring the methylation status of the T cells in the sample;
   c) establishing a multipotency score for the T cells based on a comparison of the methylation status of the T cells to a T-cell multipotency index;
   d) identifying the level of auto-reactive T cells in the sample based on the multipotency score.
73. The method of embodiment 72, wherein a multipotency score higher than a control indicates a high level of auto-reactive T cells, thereby identifying the patient as one who requires further monitoring or treatment.
74. The method of embodiment 72, wherein a multipotency score lower than a control indicates a low level of auto-reactive T cells, thereby identifying the patient as one in which T cell tolerance has been induced.
75. The method of embodiment 73 or 74, wherein the control is a T cell population obtained from the patient at a previous time point.
76. The method of embodiment 73 or 74, wherein the control is a pre-defined threshold.
77. The method of embodiment 73 or 74, wherein the control is a T cell population obtained from a healthy individual.
78. The method of any one of embodiments 72-77, wherein the autoimmune disease is type 1 diabetes.
79. The method of any one of embodiments 73-78, wherein the patient is one who has previously been administered a therapeutic to treat the autoimmune disease.
80. A method of generating a T cell multipotency index, said method comprising:
   a) isolating at least two populations of T cells;
   b) identifying CpG sites that are differentially methylated in the at least two populations of T cells; and
   c) assigning a weighted index score to each CpG site.
81. The method of embodiment 80, wherein the CpG sites are identified by supervised analysis of training data sets comprising genome-wide methylation profiles of the least two populations of T cells, wherein the at least two populations of T cells are at different stages of differentiation.
82. The method of embodiment 81, where the T cells are selected from naïve T cells, stem memory T cells, central memory T cells, effector memory T cells, or effector memory-like T cells.
83. The method of embodiment 82, wherein the effector memory-like T cells are HIV-specific CD8 T cells.
84. The method of embodiment 83, wherein the at least two populations of T cells comprise one or more populations of naïve T cells and one or more populations of HIV-specific CD8 T cells.
85. The method of embodiment 82, wherein the at least two populations of T cells comprise one or more populations of naïve T cells and one or more populations of effector memory cells.
86. The method of any one of embodiments 80-85, wherein one or more machine learning algorithm identifies the differentially methylated CpG sites and generates weighted index scores for each identified CpG site, thereby generating the multipotency index.
87. The method of embodiment 86, wherein the one or more machine learning algorithm is a one-class logistic regression algorithm.
88. The method of any one of embodiments 80-87, wherein the T cell multipotency index comprises one or more weighted index scores, and wherein each weighted index score corresponds to a CpG site in a T cell genome.
89. A kit comprising reagents for detecting a methylation status of one or more CpG sites selected from Table 1 in subject T cells, wherein the kit further includes instructions for accessing, utilizing, or generating a multipotency index.
90. The kit of embodiment 89, comprising reagents for detecting the methylation status of at least 5%, at least 10%, at least 20%, at least 30%, at least 40%, at least 50%, at least 60%, at least 70%, at least 80%, or at least 90% of the 245 CpG sites in Table 1 in the subject T cells.
91. The kit of embodiment 89, comprising reagents for detecting the methylation status of each CpG site in Table 1 in the subject T cells.
92. The kit of embodiment 89, consisting of reagents for detecting the methylation status of each CpG site in Table 1 in the subject T cells.
93. The kit of any one of embodiments 89-92, further comprising a package insert comprising instructions for accessing, utilizing, or generating a T cell multipotency index based on the methylation status of the one or more CpG sites.

### EXPERIMENTAL

### Example 1. Human Self-Reactive CD8 T Cells Acquire Stem-Cell Memory-Associated Epigenetic Programs During Development of Type 1 Diabetes

### Introduction & Results

Self-reactive T cells play an important role in the development of autoimmune diseases and are responsible for a wide spectrum of life-long immunopathologies. Often considered a quintessential autoimmune disease, Type 1 diabetes (T1D) is distinguished by the enduring capacity of self-reactive T cells to destroy insulin-secreting beta cells in the pancreatic islets of Langerhans. These self-reactive CD8 T cells retain the ability to recognize and kill beta cells in vitro, exemplifying their extraordinarily long-lived effector capacity. Furthermore, individuals that receive a pancreatic islet transplant are prone to a rapid antigen-specific effector CD8 T cell response, indicating that the beta cell-specific CD8 T cells underwent a differentiation program the enabled them to remain poised to mount a recall response. In addition to infiltrating the islets, these antigen-specific CD8 T cells are also present throughout the circulatory system of T1D patients. Based on the phenotypic characterization of beta cell-specific CD8 T cells found in circulation, recent studies have reported a correlation between disease severity and a T cell phenotype associated with limited homeostatic proliferation (namely, effector-memory or Tem). However, another study reported that the majority of beta cell-specific CD8 T cells possess a less differentiated stem-cell memory (scm) phenotype. The discrepancy between these phenotypic analyses raises many unresolved questions about the differentiation status of these cells. Hence, there is a critical need to elucidate the molecular mechanisms regulating the development and maintenance of effector and memory T cell-associated properties of human beta cell-specific CD8 T cells.

Broadly, epigenetic modifications, which include histone modifications and DNA methylation, influence gene expression patterns without altering the underlying DNA sequence. By providing a mechanism to heritably propagate acquired gene expression programs in a dividing population of cells, epigenetic modifications can be utilized to reinforce cell fate decisions. This concept has been applied to describe the long-lived effector potential of memory CD8 T cells following infection. Our group and others have recently demonstrated a causal relationship between epigenetic programming and the maintenance of effector and memory-associated functions during T cell homeostasis to sustain long-lived immunity. During the development of long-lived memory CD8 T cells, activated naïve antigen-specific CD8 T cells transition through the effector stage of differentiation, enabling a subset of cells to acquire effector-associated programs prior to their continued development into memory CD8 T cells. The transient exposure to effector-promoting signals imparts memory T cells with long-lived effector-associated gene expression that endow memory T cells with a heightened ability to recall effector functions while retaining the naïve-like capacity to develop into other memory and effector cell types. Importantly, the blend of naïve and effector properties among memory CD8 T cells is reflected by their epigenetic profiles being similar to both naïve and effector T cells.

Building upon these recent insights, we proceeded to define the epigenetic landscape of beta cell-specific CD8 T cells to better understand the barriers for tolerizing self-reactive T cells. Here, we applied the concept that changes in DNA methylation reinforce CD8 T cell fate decisions and generated an epigenetic atlas that can predict human CD8 T cell differentiation status. Using this novel bioinformatic tool, we investigated the relationship between epigenetic programs and the longevity of human autoreactive T cell responses during T1D. Epigenetic characterization of MHC class I tetramer+ beta cell-specific CD8 T cells isolated from the circulation of type 1 diabetics revealed that this pool of autoreactive T cells is imparted with epigenetic programs associated with both naïve and effector-associated properties. The coexistence of both naive and effector epigenetic programs in beta cell-specific CD8 T cells was further interrogated using single cell ATAC-seq, revealing that beta cell-specific CD8 T cells exhibit transcriptionally permissive regions consistent with both naïve and effector stages of differentiation. We further validated our findings by establishing a murine version of the multipotency index and characterizing endogenous beta cell-specific CD8 T cells from lymphoid tissues and the pancreas of NOD mice. Consistent with the results from our human self-reactive T cell epigenetic analyses, mouse beta cell-specific CD8 T cells isolated from tissues away from the source of antigen retain a stem-like epigenetic state. Collectively, the results presented here indicate that beta cell-specific CD8 T cells can acquire a hybrid of naïve and effector associated epigenetic programs and provides a mechanism to explain how the stem-like state of the cells can sustain the autoreactive immune response.

Beta Cell-Specific CD8 T Cells Acquire Tscm-Like Epigenetic Programming

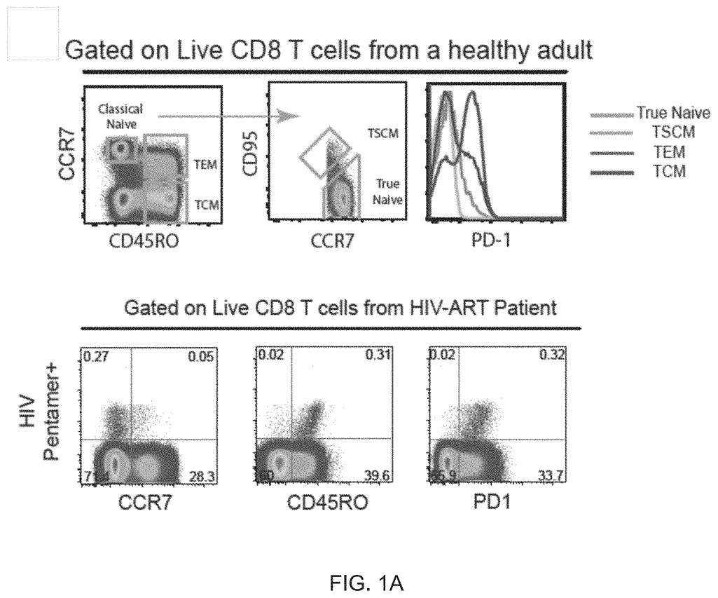

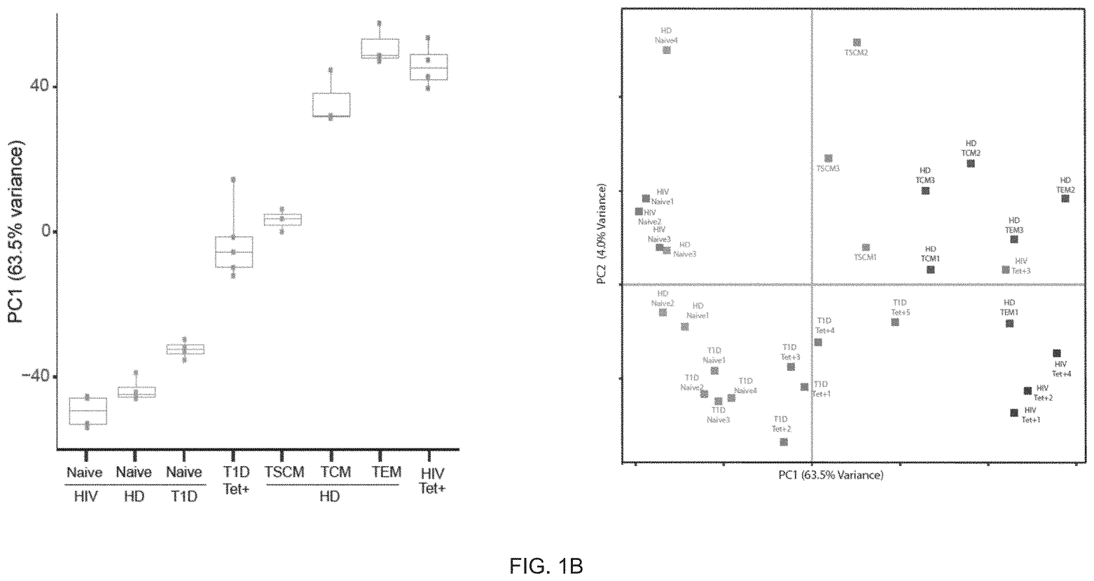

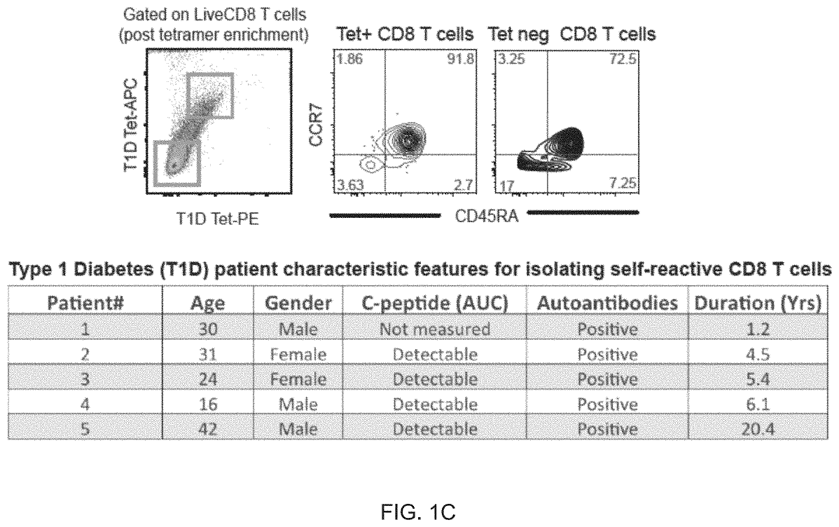

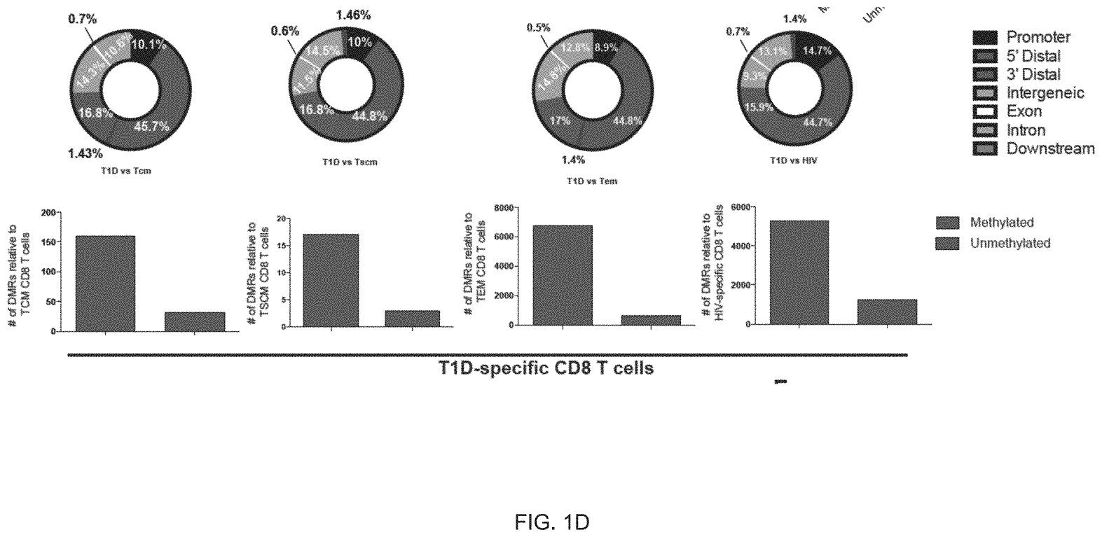

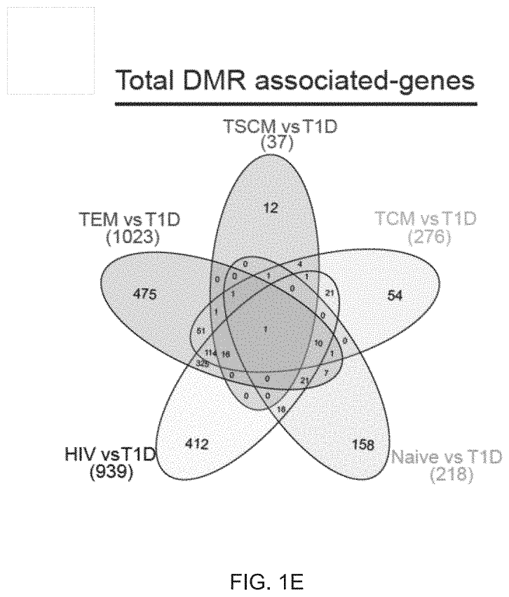

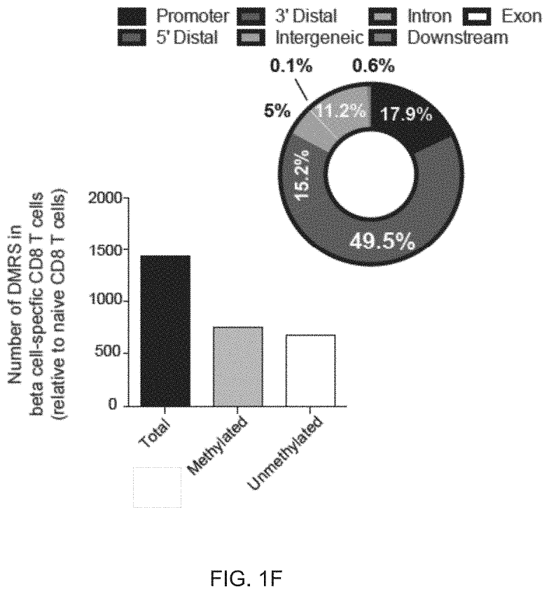

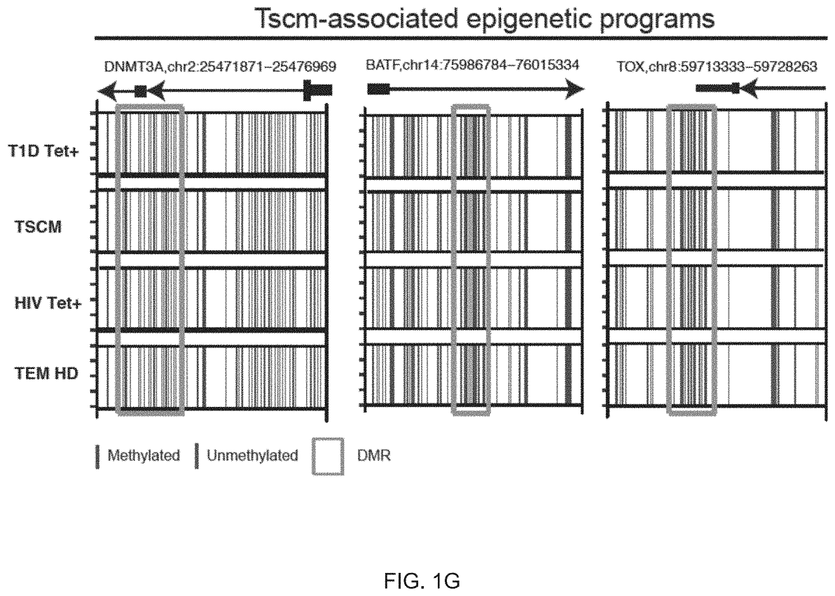

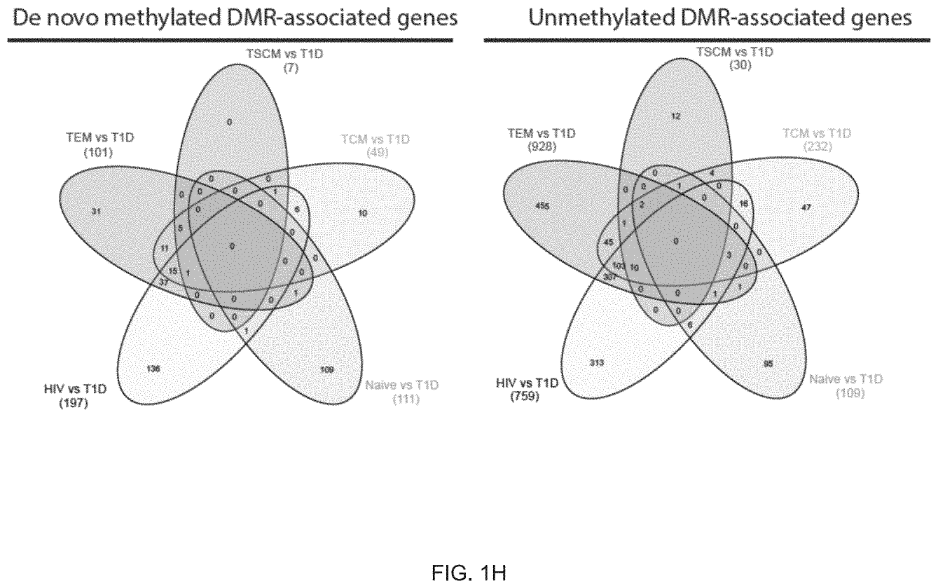

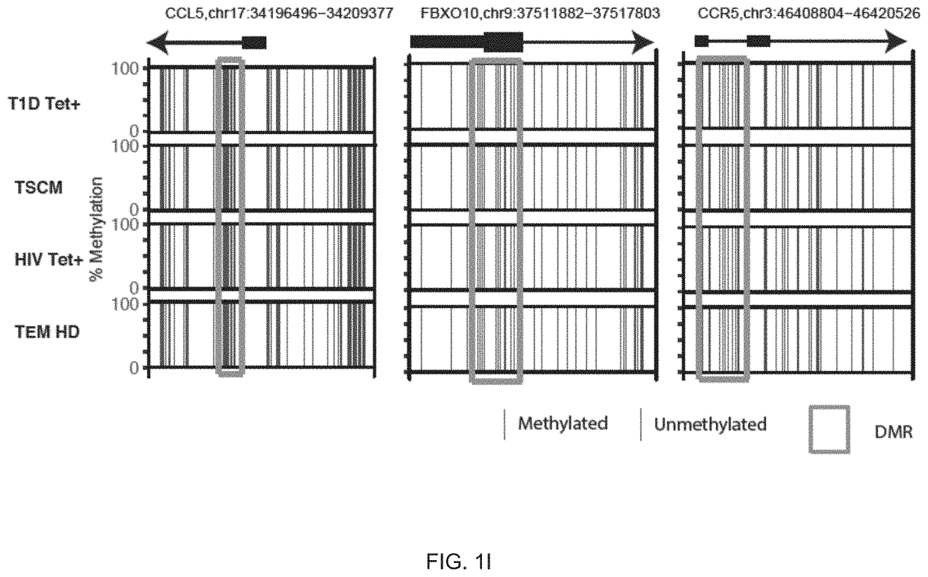

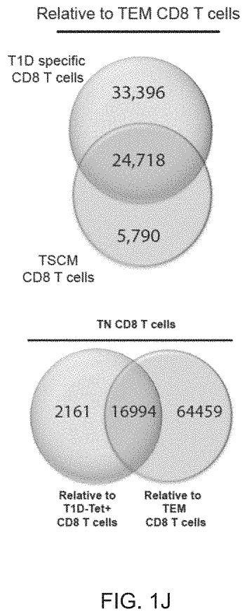

In order to fully contextualize the differentiation-associated programs among beta cell-specific T cells, we first generated an epigenetic atlas of human CD8 T cell differentiation. To establish a broad spectrum of human CD8 T cell differentiation-associated epigenetic profiles, we generated whole-genome bisulfite sequencing (WGBS) DNA methylation profiles from naïve, short-lived, and long-lived memory CD8 T cells (FIG. 1A-upper panel). These polyclonal CD8 T cell subsets, isolated from healthy individuals, cover a developmental spectrum ranging from less-differentiated (Naïve and Tscm) to more differentiated CD8 T cells (Tem). In addition to the polyclonal CD8 T cell subsets, we also generated DNA methylation profiles of HIV-specific CD8 T cells from chronically infected individuals to characterize DNA methylation profiles of cells that have undergone differentiation in the setting of chronic antigen exposure. The HIV-specific CD8 T cells exhibited phenotypic properties similar to Tem cells but also expressed high levels of the inhibitory receptor PD-1(FIG. 1A-bottom panel), which is epigenetically reinforced even following therapeutic reduction in viral load. Taken together, these data indicate that the HIV-specific T cells are representative of a population of cells that have undergone differentiation in response to a chronic source of antigen and provide a benchmark for terminal differentiation in our analysis.

Having generated whole-genome, nucleotide-resolution methylation profiles of three or more biological replicates for each of these T cell populations, we next performed an unsupervised principal component analysis (PCA) of the CpG methylation status involving the most variable 3000 CpG sites across the genome. Notably, PC1 explained >60% of the variance among all samples. Importantly, the greatest segregation between samples was among the naïve compared to Tem and HIV-specific CD8 T cells, consistent with these subsets providing a lower and upper bounds on the developmental spectrum, respectively. Furthermore, this analysis revealed a notable clustering of naïve CD8 T cells isolated from both HIV-infected individuals and healthy individuals. Lastly, the PCA placed the most developmentally plastic memory T cell population, Tscm, in an area of this “epigenetic spectrum” that is approximately equidistant between naïve and chronically stimulated virus-specific T cells (FIG. 1B). Broadly, this analysis documents the linkage between DNA methylation programming and the putative multipotent capacity of human memory CD8 T cells as well as provides a framework to further characterize beta cell-specific CD8 T cells.

With an epigenetic atlas of human T cell differentiation established, we proceeded to generate whole-genome DNA methylation profiles of beta cell-specific CD8 T cells from a well-defined cohort of T1D patients with a range in disease duration spanning 1 to 20 years (FIG. 1C bottom table). After generating five independent whole-genome DNA methylation profiles of beta cell-specific CD8 T cells, we again performed PCA of all the whole-genome DNA methylation profiles. To our surprise, beta cell-specific T cell populations from all five participants, regardless of disease duration, clustered most closely to the Tscm CD8 T cells (FIGS. 1B-1D). Notably, this analysis revealed that the self-reactive T cells isolated from the circulation of T1D patients were strikingly segregated from T cells that had progressed to a more terminally differentiated state (i.e., chronically stimulated HIV-specific CD8 T cells) (FIG. 1B). Thus, despite the persistent antigen exposure and T cell activation that occurs in T1D, beta cell-specific CD8 T cells in the circulation of T1D do not acquire an epigenetic program associated with prolonged TCR engagement. Rather, beta cell-specific CD8 T cells acquire an epigenetic program associated with the multipotent capacity of Tscm CD8 T cells.

To further define the DNA methylation programs associated with the developmental status of self-reactive CD8 T cells, we performed a pairwise comparison of gene-associated differentially methylated regions (DMRs) between beta cell-specific CD8 T cells and each individual CD8 T cell subset in addition to HIV-specific CD8 T cells. Notably, Tscm and beta cell-specific CD8 T cells had the most similar DNA methylation profiles with only thirty-seven DMR-associated genes (FIG. 1E). Further inspection of other DMR pairwise comparisons demonstrated a significantly greater number of differences in gene-associated DNA methylation programs between the T1D self-reactive CD8 T cells and Tcm, Tem, and HIV-specific T cell populations. In particular, self-reactive T cells are often described as sharing features with naïve CD8 T cells and exhibiting a similar CD45RA+ CCR7+ phenotype (FIG. 1C). Therefore, we proceeded to interrogate potential differences among the naïve and beta cell-specific DNA methylation profiles. We identified more than 1000 regions that were differentially methylated between the naïve and self-reactive T cells, encompassing both de novo DNA methylation and demethylation events (FIG. 1F, bottom panel). Importantly, most (˜95%) of these DMRs were located at or near genes (FIG. 1F, top panel). Broadly, these data document methylation programs that distinguish self-reactive CD8 T cells from CD8 T cells in other differentiation states and highlight the epigenetic similarity between beta cell-specific CD8 T cells and Tscm.

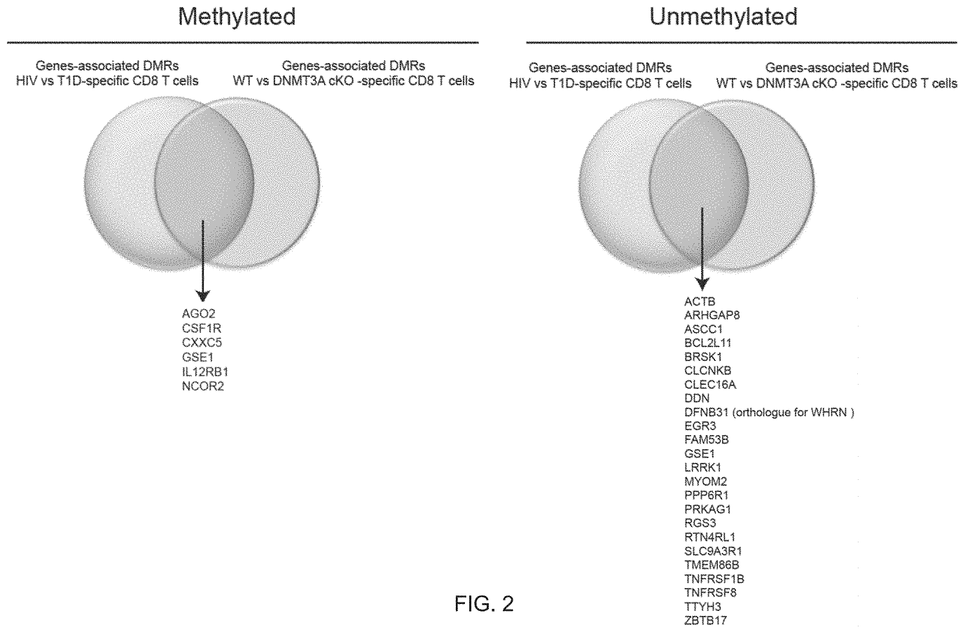

The marked overlap in DNA methylation programming among Tscm and self-reactive T cells prompted us to further characterize methylation programs that are specifically shared between these populations that delineate these cells from other differentiated populations (FIG. 1D; FIGS. 1F-1J). Shared methylation programs included regions located within TOX, DNMT3A, and BATF loci. Importantly, each of these transcription factors are critically involved in the terminal differentiation of T cells. Specifically, the TOX locus, a transcription factor recently shown to play a critical role in the survival of exhausted T cells, was demethylated in Tem and HIV-specific CD8 T cells and remained methylated in both Tscm and the beta cell-specific CD8 T cells. Additionally, consistent with its critical role in regulating the self-renewal capacity of embryonic and hematopoietic stem cells, we observed that the DNMT3a internal promoter was significantly methylated in more differentiated T cell populations and remained unmethylated in the Tscm and self-reactive T cells. Notably, expression of DNMT3a isoform 2 has been historically associated with stem cells. Because of the unmethylated status of the DNMT3a internal promoter, we cross referenced the previously defined Dnmt3a targeted loci in murine T cells with our existing Tem and HIV-specific CD8 T cell DMR-associated gene list. Indeed, many of the genes targeted for Dnmt3a-mediated methylation in terminally differentiated mouse T cells were also differentially methylated among the self-reactive versus chronically stimulation virus-specific CD8 T cells (FIG. 2). Lastly, the gene body of B cell activating factor (BATF), a transcription factor involved in the self-renewal of hematopoietic stem cells, was highly methylated in beta cell-specific and Tscm CD8 T cells but unmethylated in the more differentiated CD8 T cells (FIG. 1G). Collectively, these data indicate that differentiation of beta cell-specific CD8 T cells is coupled to an epigenetic rewiring of the cell that may preserve its multipotent capacity.

Development and Application of a Human DNA Methylation-Based T Cell Multipotency Index to Assess Beta Cell-Specific CD8 T Cell Plasticity

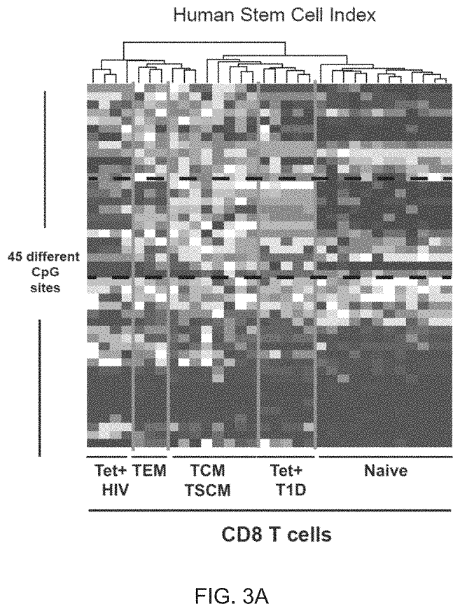

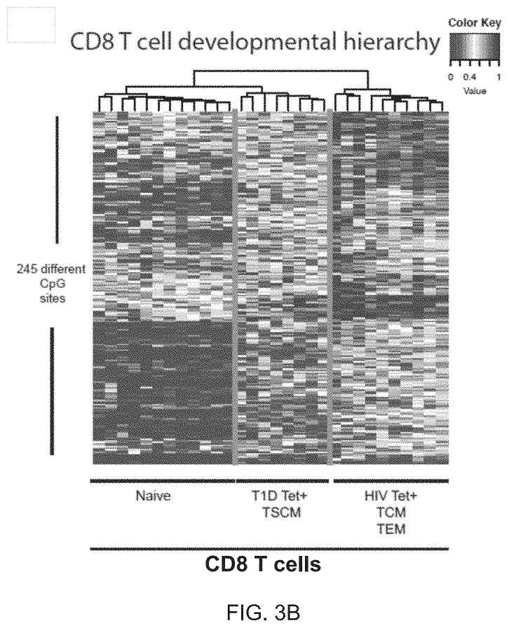

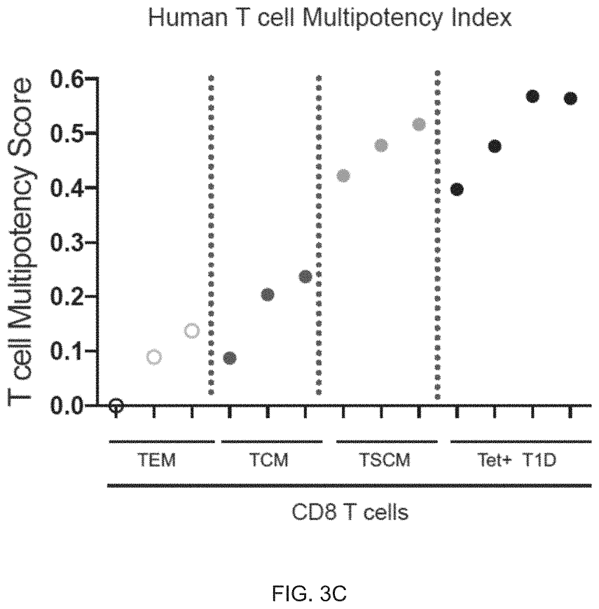

To more broadly characterize the “multipotency-associated” epigenetic status of beta cell-specific T cells, we next compared our datasets to a recently reported human stem-cell epigenetic index. This index, derived from a machine learning algorithm that interrogated gene expression and epigenetic signatures of bona fide stem-cells, consists of 219 CpG sites used to assign a “stemness” hierarchy among stem and tumor cells (see, e.g., Malta et al., Cell 173:338-354 e315 (2018)). From this index only 45 CpG sites were differentially methylated between human CD8 T cells across differentiation states (FIG. 3A). Therefore, to better define a CD8 T cell-specific multipotency index we utilized the same machine learning approach with naïve and HIV-specific CD8 T cells as training data sets to identify a set of 245 CpG sites whose methylation status can delineate the developmental hierarchy among CD8 T cells (FIG. 3B & Table 1). Dendogram representation of these data show that the beta cell-specific and Tscm CD8 T cells were nearest to the naïve CD8 T cells, with both groups being segregated from the HIV-specific and polyclonal Tem CD8 T cells. This developmental hierarchy was then normalized on a scale of 0 to 1 to generate a human T cell “multipotency index.” Previously defined methylation profiles of CD8 T cell subsets (Tem, Tcm, and Tscm) were used to validate this tool. As expected, Tem had the lowest score followed by Tcm and Tscm which is consistent with their respective levels of differentiation (with lower scores indicative of a more terminally differentiated state). Importantly, beta cell-specific CD8 T cells were found to have the highest (most naïve-like) multipotency score (FIG. 3C). Collectively, these data indicate that beta cell-specific CD8 T cells acquire an epigenetic program that is associated with retention of a T cell stem-like differentiation state. Given that the duration of disease for several of the individuals used for establishing beta cell-specific CD8 T cell methylation profiles spanned several years, these data suggest that long-term pathology may be coupled to the ability of these stem-like, self-reactive CD8 T cells to retain the potential to mount an effector response.

Beta Cell-Specific CD8 T Cells Acquire Effector-Associated Epigenetic Programs

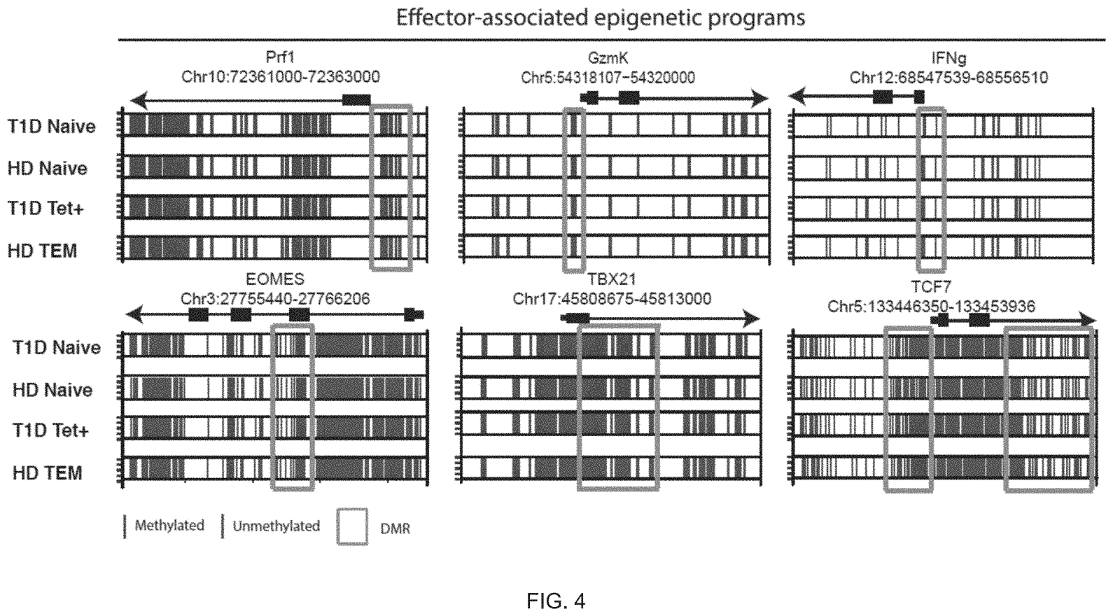

Because progressive destruction of pancreatic islet cells relies largely on the effector functions of T cells (see, e.g., Knight et al., Diabetes 62:205-213 (2013); Burrack et al., Front Endocrinol (Lausanne) 8:343 (2017)), we next interrogated the beta cell-specific T cells for effector-associated DNA methylation programs. While our initial pairwise analysis of gene-associated DMRs documented a similarity between Tscm and self-reactive T cell methylation profiles, it is important to note that the beta cell-specific T cells also acquired a core set of DNA methylation programs that were present in all memory T cell subsets. Indeed, >900 of the beta cell-specific CD8 T cell-associated DMRs, programs that delineate the self-reactive T cells from naïve CD8 T cells, were shared between Tem and/or HIV-specific CD8 T cells (FIG. 1F). These DMRs were primarily enriched in the 5′ distal regions, suggesting an association with transcriptional regulatory regions (FIG. 1D). Core programs included demethylation of loci associated with effector responses such as Perforin (Prf1), GzmK, and IFNg. These loci were predominately unmethylated in beta cell-specific CD8 T cells relative to loci in naïve CD8 T cells suggesting that they had undergone a differentiation program that enables an effector response (FIG. 4). In addition to demethylation of effector-associated loci, we observed differences in the methylation status at the loci of the transcription factors for Eomesodermin (Eomes), T-bet (Tbx21), and T cell-specific transcription factor 7 (TCF7, encoding TCF1 protein), all of which have crucial roles in the differentiation of effector and memory CD8 T cells (FIG. 4). Thus, while beta cell-specific CD8 T cells retain many epigenetic features of a less differentiated cell, the modification of effector-associated loci indicates that the cells undergo some degree of effector differentiation.

Naïve and Effector-Associated Epigenetic Programs Co-Exist in Individual Beta Cell-Specific CD8 T Cells

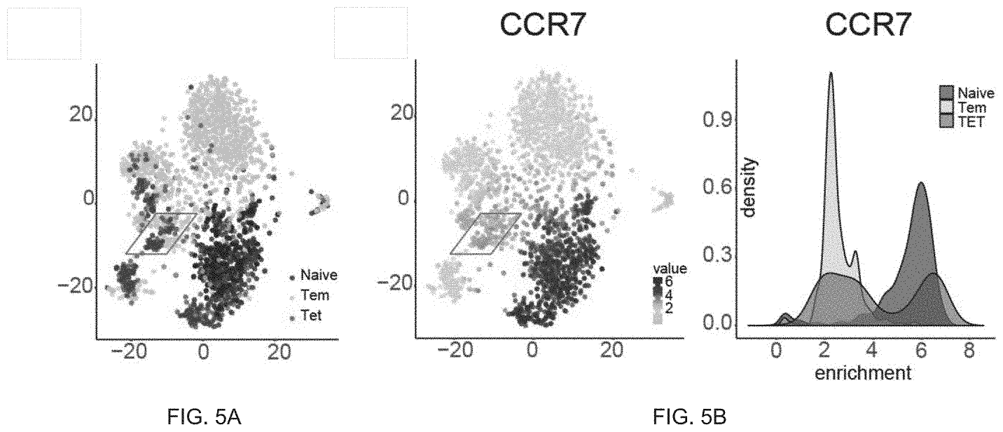

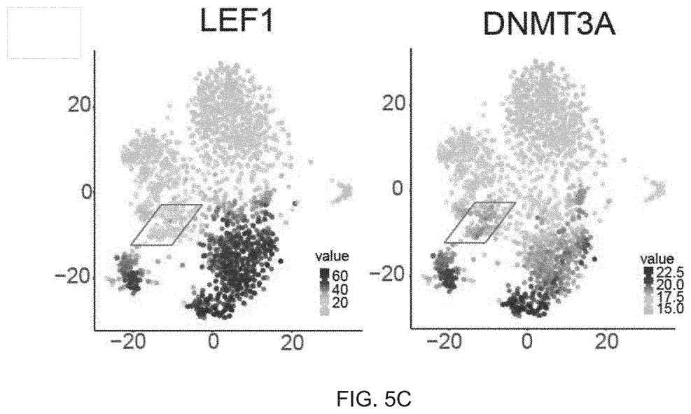

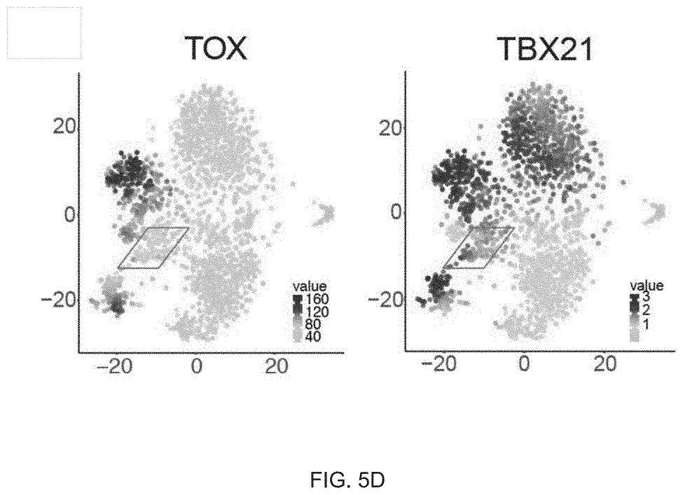

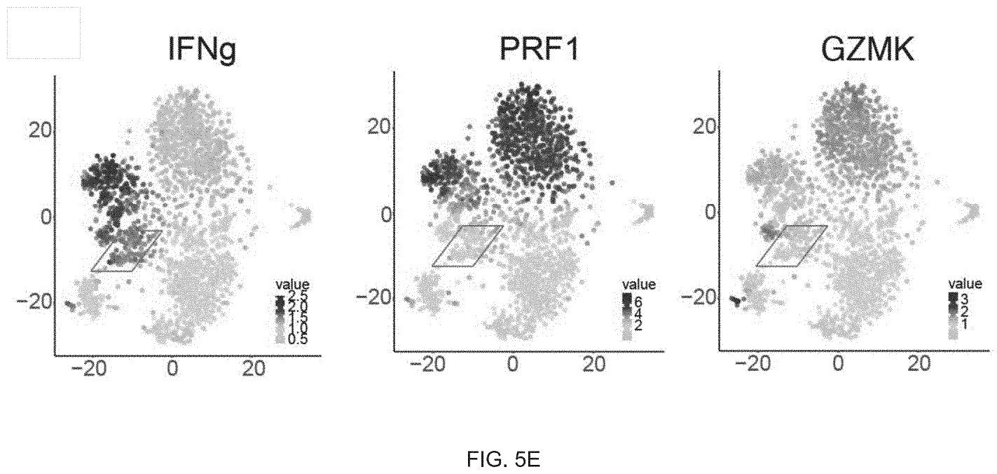

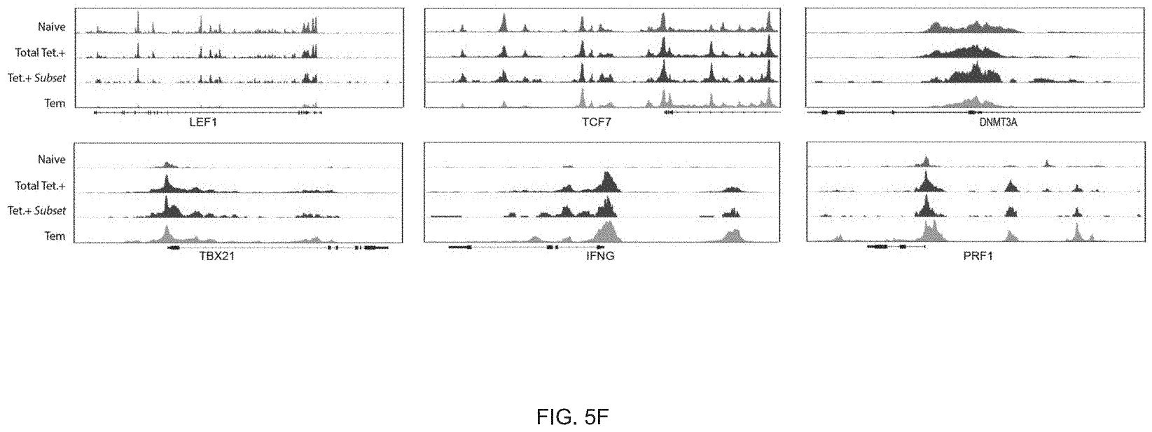

Our DNA methylation data broadly suggest that beta cell-specific CD8 T cells exhibit both naïve and effector epigenetic programs. However, because our whole-genome DNA methylation profiling approach was performed on a pool of cells, we cannot fully resolve whether the hybrid profiles are truly co-existing in individual cells or if these data result from our analyses being performed on a heterogeneous population of cells that have distinct naïve and effector-associated epigenetic programs. To further investigate the epigenetic programs acquired in individual beta cell-specific CD8 T cells, we performed single cell ATAC-seq (scATAC-seq). Tetramer positive beta cell-specific CD8 T cells, as well as donor-matched naïve and Tem CD8 T cells, were sorted from three T1D donors and scATAC-seq profiling was performed. TSNE analysis of individual cell chromatin accessibility profiles for all samples broadly documented that a large portion of the beta cell-specific CD8 T cells resemble a transition state between the naïve and Tem subsets (FIG. 5A), which is similar to our results from the DNA methylation profiling studies shown in FIG. 1B. Interestingly, a subpopulation of beta cell-specific CD8 T cells (highlighted in green) exhibits hybrid properties of both naïve and Tem subsets. To confirm that the accessibility profiles indeed delineate the naïve and effector-like states, we examined the accessibility of the CCR7 locus which is known to acquire epigenetically repressive modifications in Tem CD8 T cells (see, e.g., Abdelsamed et al., J Exp Med 214:1593-1606 (2017)). Indeed, all naïve CD8 T cells had high levels of chromatin accessibility at the CCR7 locus while this locus was largely inaccessible among the pool of Tem CD8 T cells (FIG. 5B). Furthermore, interrogation of the CCR7 locus revealed that a population of beta cell-specific CD8 T cells had an accessibility profile that suggested a transitional state between both the naïve and Tem subsets. We next proceeded to interrogate the accessibility profiles of individual effector and naïve-associated loci among the beta cell-specific CD8 T cells. Importantly, we observed distinct subsets of tetramer+ beta cell-specific CD8 T cells based on these profiles. Notably, one subset of tetramer+CD8 T cells was indistinguishable from naïve CD8 T cells. However, a separate population of tetramer+CD8 T cells segregated from the majority of both naïve and Tem CD8 T cells. Focusing on this subset of beta cell-specific CD8 T cells, we examined several genes that were representative of the hybrid naïve and effector state observed in the DNA methylation data set. These loci included the stem-associated transcriptional regulators Tcf7, Lef1, and DNMT3a, effector/activation-associated transcription factors Tbet and Tox, and effector molecules, IFNg, Prf1, and GzmK. Notably, this cluster of beta cell-specific CD8 T cells was found to have accessible chromatin at the loci of the transcriptional activators of the effector/chronic stimulation T response (Tbet and Tox) and the accompanying effector molecules (IFNg and Gzmk) (FIGS. 5C-5F). Quite strikingly, this same cluster of cells retained accessibility at the stem-associated loci of LEF1 and DNMT3a (FIGS. 5C-5F). These data document the coexistence of both naïve and effector-associated epigenetic programs among individual beta cell-specific CD8 T cells. While many of the beta cell-specific CD8 T cells overlap with either the naïve and Tem, the existence of a hybrid tetramer+ cell population further supports the idea that the sustained autoreactive state of these T cells may be preserved by a long-lived population of cells that retain effector potential. These data further demonstrate the utility of the epigenetic-based multipotency index (DNA methylation profiling) to predict the developmental plasticity of human T cells.

Beta Cell-Specific CD8 T Cells Maintain Stemness-Associated Epigenetic Programs During In Vitro Expansion

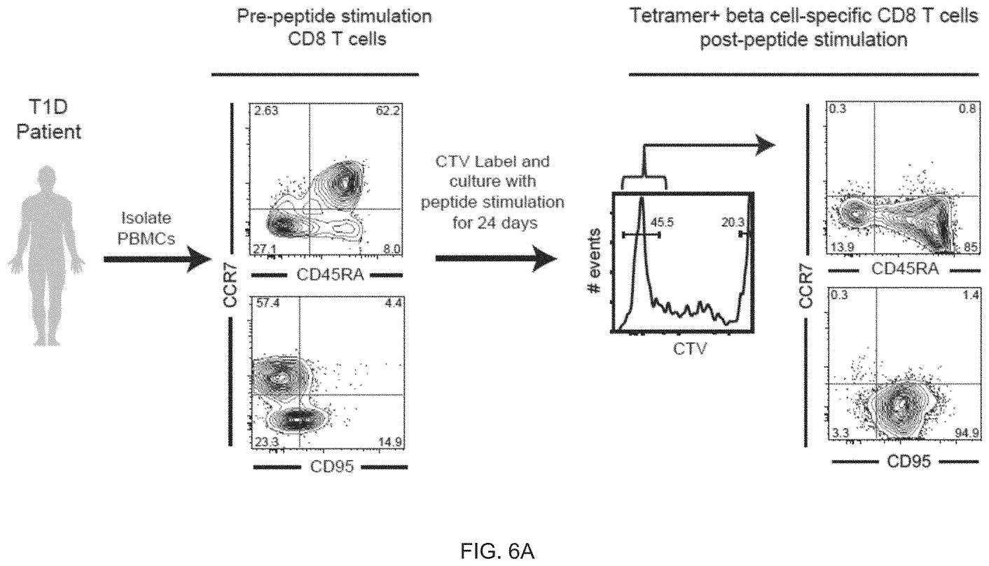

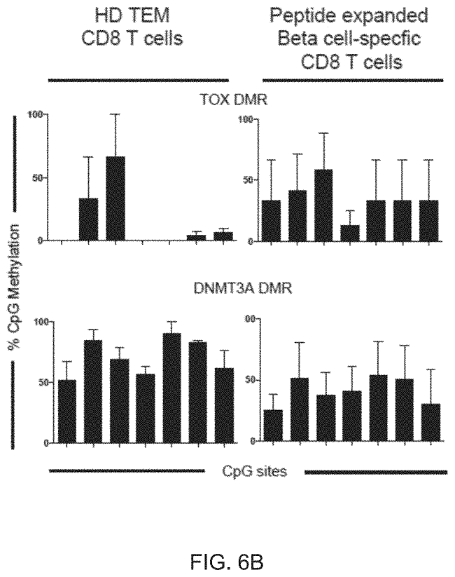

A hallmark of T1D is the prolonged effector response of T cells during prolonged antigen exposure. Our data suggest that the longevity of the effector T cell response in T1D patients is coupled to the ability of self-reactive CD8 T cells to preserve their effector capacity during prolonged stimulation while also maintaining a developmentally plastic state of differentiation. To test the stability of multipotency-associated programs, we proceeded to determine if these programs were retained during ex vivo antigen-driven expansion of beta cell-specific CD8 T cells. Peripheral blood mononuclear cells (PBMCs) were obtained from a T1D patient and then labeled with cell trace violet (CTV), a dye to track cell proliferation. The CTV-labelled cells were then cultured with a mixture of peptides recognized by T1D self-reactive CD8 T cells. After 14-24 days of ex vivo culture, tetramer positive CD8 T cells that underwent several rounds of proliferation were phenotypically characterized and FACS-purified for subsequent DNA methylation analyses (FIG. 6A). Notably, the divided population of cells expressed high levels of CD95 and CD45RA and had downregulated CCR7 (FIG. 6A), a phenotype often ascribed to an effector response. We next proceeded to determine if these cells retained the multipotency-associated epigenetic programs using DMRs at TOX and DNMT3A loci as surrogates of such programs. Importantly, targeted methylation profiling of these loci revealed that these programs remained unchanged during ex vivo antigen-dependent proliferation of beta cell-specific CD8 T cells: the TOX locus remained partially methylated in beta cell-specific CD8 T cells compared to mostly unmethylated Tem, while the DNMT3A locus remained partially methylated compared to the mostly methylated Tem (FIG. 6B). These data collectively demonstrate that despite having a significant proportion of cells exhibiting a Tem phenotype, beta cell-specific CD8 T cells maintain multipotency-associated epigenetic programming after undergoing extensive antigen-dependent proliferation.

Stem-Associated Epigenetic Programs are Enriched in Beta Cell-Specific CD8 T Cells Isolated from Circulation and Lymphoid Tissue

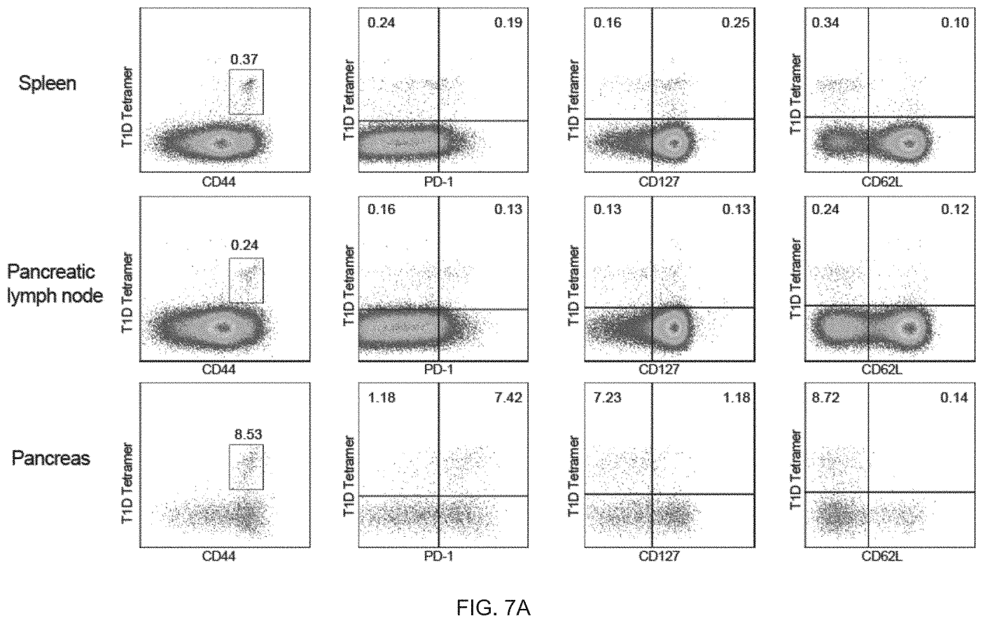

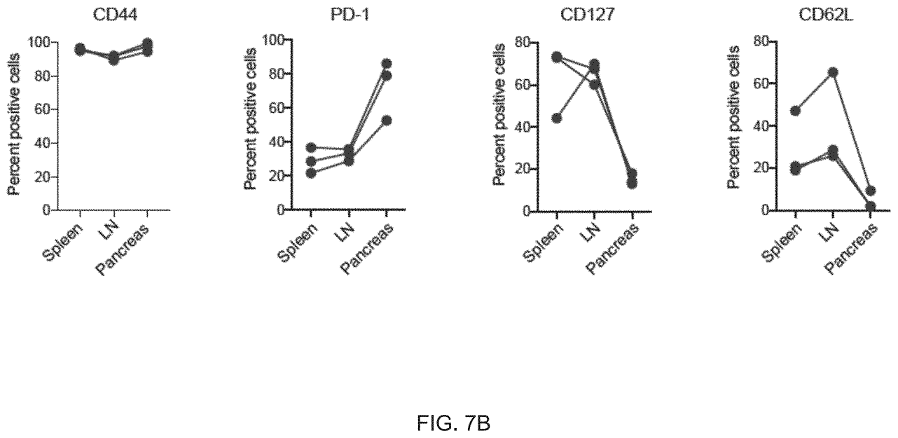

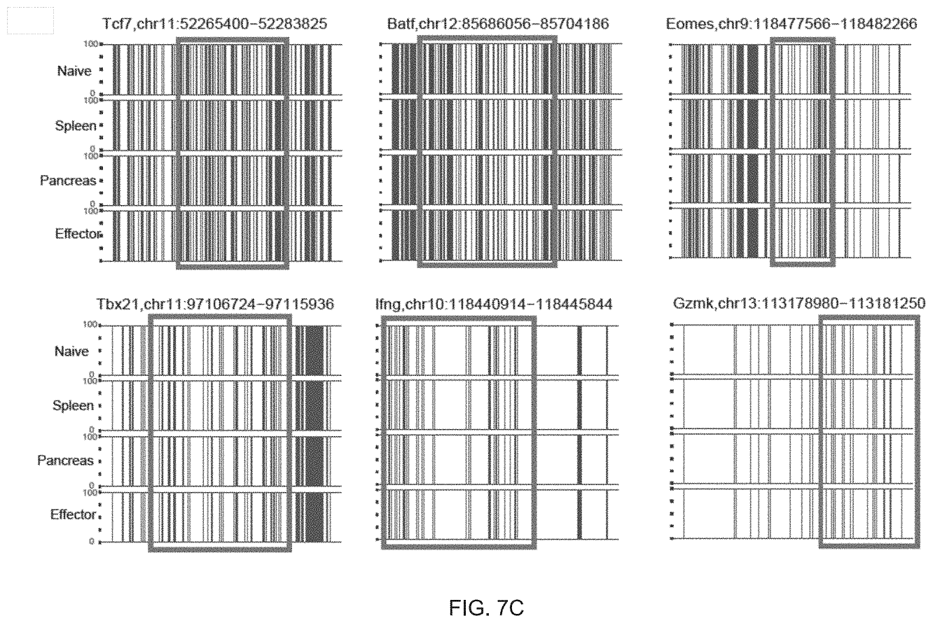

Given that human beta cell-specific CD8 T cells isolated from the peripheral blood of individuals with T1D retain stem-associated DNA methylation programs following in vitro expansion, we sought to investigate whether the developmentally plastic state of self-reactive CD8 T cells was also preserved in cells that traffic to the source of the antigen. To overcome the challenge of examining patient beta cell-specific T cells from various anatomical locations, we utilized an established murine model that allowed us to examine an endogenous immune response in a site-specific manner (see, e.g., Garyu et al., J Biol Chem 291:11230-11240 (2016)). Tetramer+ beta cell-specific CD8 T cells were sorted from spleen, pancreatic lymph node, and pancreas of non-obese diabetic (NOD) mice and whole-genome DNA methylation profiling was performed. Notably, the phenotype of the beta cell-specific CD8 T cells isolated from the lymphoid tissues versus the pancreas were strikingly distinct. Lymphoid residing beta cell-specific CD8 T cells retained the phenotype of a multipotent memory T cell depicted by higher expression of CD127 and CD62L and lower PD-1 expression. Conversely, beta cell-specific CD8 T cells isolated directly from the pancreas exhibited an effector phenotype with low expression of CD127 and CD62L and higher expression of PD-1 (FIGS. 7A and 7B). Importantly, the phenotypic differences associated with the anatomical location were also consistent with the cell's differentiation-associated epigenetic status. For instance, beta cell-specific CD8 T cells isolated from the pancreas possess an effector-like methylation pattern at the TCF7 locus while the same antigen-specific CD8 T cells isolated from the spleen have a naïve-like pattern (FIG. 7C). A similar pattern is seen at the IFNg locus with antigen-specific CD8 T cells isolated from the pancreas exhibiting an effector-like methylation pattern compared to the more naïve-like pattern in the spleen-derived antigen-specific T cells. While distinct methylation patterns are observed between the spleen- and pancreas-derived beta cell-specific CD8 T cells at several key loci, differences between the two cell populations were not uniformly observed at all gene loci. Examination of other relevant loci such as Batf, Eomes, Tbet (Tbx21), and Gzmk revealed epigenetic programming most consistent with an effector-like cell population. Collectively, these data are consistent with our findings from human beta cell-specific CD8 T cells isolated from the peripheral blood of type 1 diabetics indicating that both murine and human beta cell-specific CD8 T cells in the periphery retain a hybrid of naïve and effector epigenetic programs. Further, these results indicate that the beta cell-specific CD8 T cells may lose their naive-like state as they traffic to the site of antigen.

Differentiation Status of Beta-Cell Specific CD8 T Cells is Dependent Upon the Site of Antigen Exposure

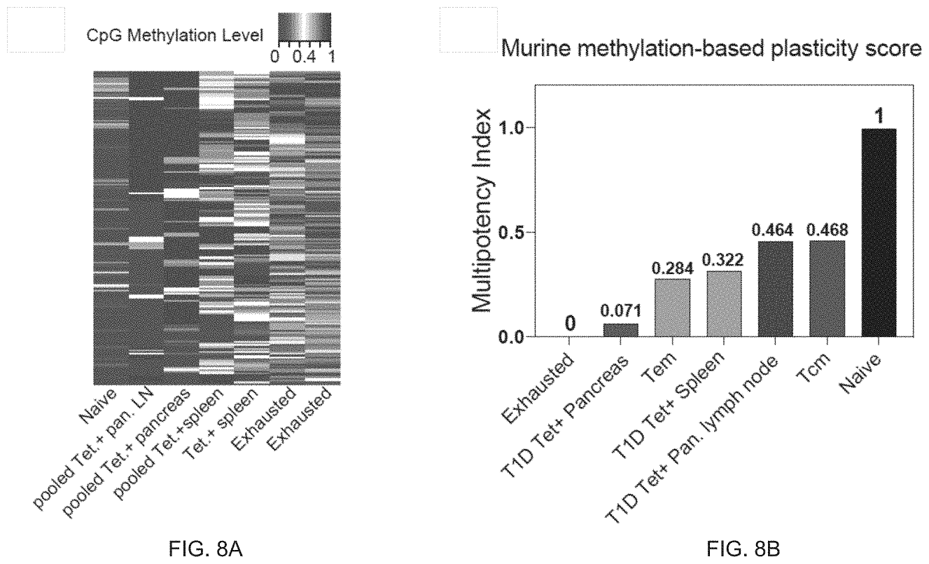

To further resolve whether a plasticity-associated epigenetic program delineates lymphoid-residing versus pancreas-residing beta cell-specific CD8 T cells, we developed a murine-version of the CD8 T cell multipotency index and applied it to the antigen-specific CD8 T cell whole-genome DNA methylation data sets. This index utilized the same machine learning approach as the human multipotency index but was derived using training data sets with murine naïve and LCMV-specific exhausted CD8 T cells. This approach identified 177 CpG sites whose methylation status can delineate the developmental hierarchy among murine CD8 T cells (FIG. 8A). Application of this murine multipotency index to our beta cell-specific CD8 T cells documented that pancreas-derived CD8 T cells have the lowest multipotency index while spleen-derived and pancreatic lymph node-derived CD8 T cells have a multipotency index comparable to long-live LCMV-specific memory T cells (FIG. 8B). Broadly, these data are consistent with our results from human beta cell-specific CD8 T cells demonstrating that the circulating autoreactive T cells acquire an epigenetic program associated with retention of a less differentiated state. Interestingly, beta cell-specific CD8 T cells derived from murine pancreas have a lower multipotency score than Tem indicating they are more terminally differentiated, a cell fate consistent with their effector-like phenotype and epigenetic programs. Taken together, these data indicate that preservation of the auto-reactive potential of beta cell-specific CD8 T cells is coupled to the acquisition of an epigenetic program that preserves a multipotent developmental potential in both mouse and human T1D. In addition to providing new insight into the disease etiology of T1D, our studies also highlight the general applicability of the methylation-based multipotency index to interrogate the developmental plasticity of CD8 T cells derived from both humans and mice. Moreover, this novel tool may be broadly applied to interrogate the differentiation status of T cells from a wide range of disease settings as well as therapeutic modalities that utilize T cells in adoptive transfer settings.

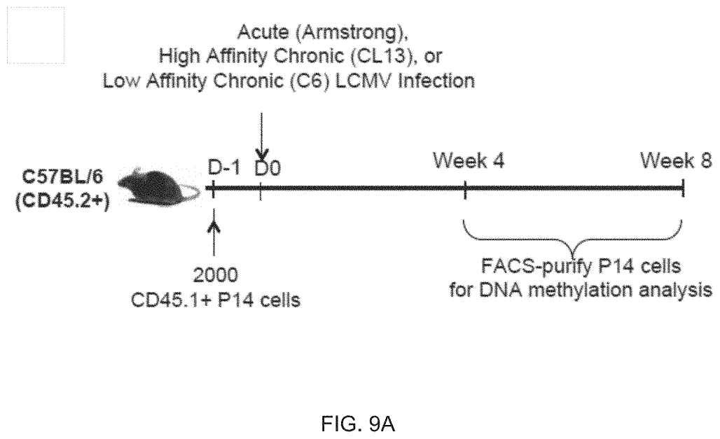

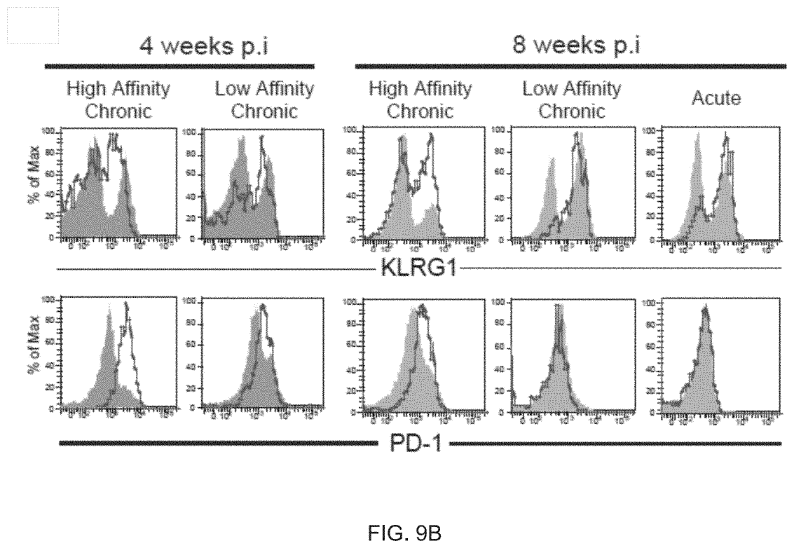

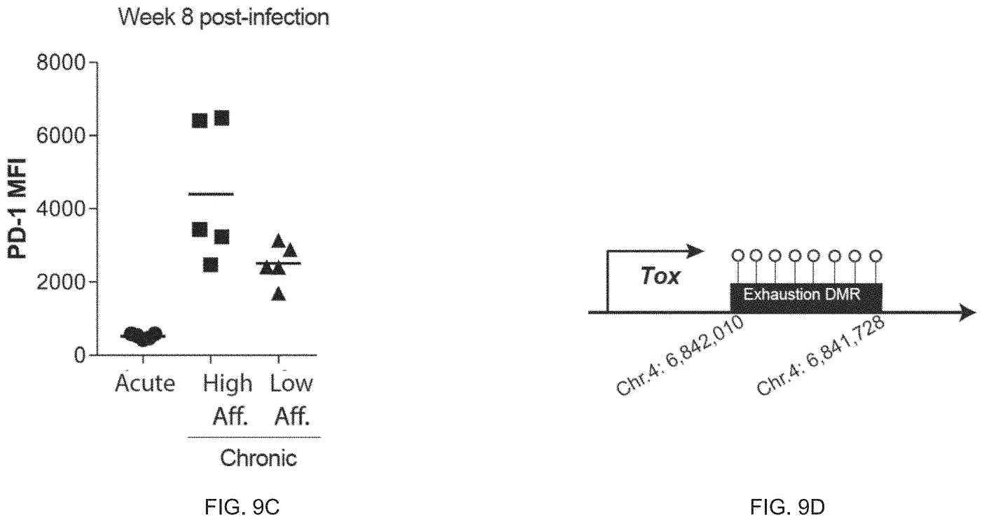

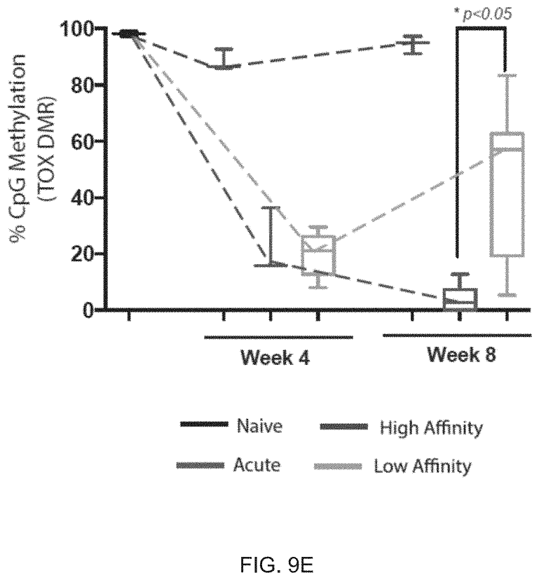

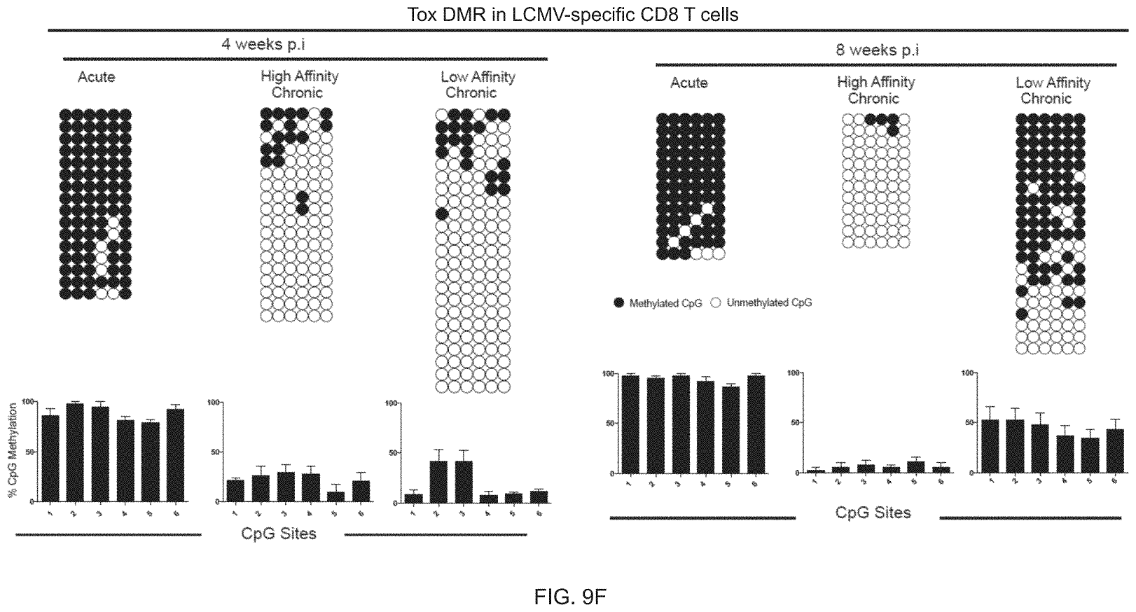

In contrast to the progressive suppression of effector functions that occurs among chronically-stimulated viral or tumor-specific T cells, beta cell-specific CD8 T cells resist suppression of their effector response. While there may be many contributing factors that enable self-reactive T cells to resist progression to a more terminally differentiated state, several lines of evidence suggest that the low binding affinity of T1D autoantigens limit the stimulation that these T cells experience. To test this hypothesis, we adoptively transferred 2×10³ congenically-distinct P14 cells (TCR transgenic CD8 T cells specific to lymphocytic choriomeningitis virus [LCMV]-specific gp33 epitope) into C57BL/6 mice followed by infection with acute LCMV-Armstrong, wild-type chronic LCMV-Clone 13, or a mutant strain of Clone 13 (C6) that has lower TCR binding affinity of P14 cells to the gp33 epitope. Acute infection with LCMV-Armstrong was used to generate highly functional, long-lived memory T cells as a comparison. Phenotypic assessment of P14 cells occurred four and eight weeks post-LCMV infection and were subsequently FACS-purified for DNA methylation analyses (FIG. 9A). P14 cells isolated from both cohorts of CL13- and C6-infected animals expressed high levels of PD-1, consistent with high the viral loads identified in these animals (FIGS. 9B-9C), thus confirming that both sets of P14 cells used for the methylation studies were chronically stimulated. We next examined the methylation status of the TOX locus (FIGS. 9D-9E). Notably, the TOX locus methylation status in murine long-lived memory T cells generated from Armstrong acute infection mirrored the status we previously observed in the human T cell subsets in which the locus remained fully methylated. Furthermore, P14 cells isolated from mice infected with CL13 had a fully unmethylated region in the TOX locus while this region remained partially methylated in isolated P14 cells from mice infected with low affinity LCMV mutant strain, C6 (FIGS. 9E-9F). Beyond suggesting that T cells stimulated with chronic, low-binding affinity TCR epitopes can retain epigenetic programs associated with developmental plasticity, these data also provide a potential mechanistic explanation of how chronically stimulated self-reactive T cells can resist acquisition of terminal differentiation programs and contribute to long-lived pathogenesis.

### Discussion

It is generally accepted that the severity and life-long nature of T1D is intimately coupled to a failure in establishing central and/or peripheral tolerance among self-reactive T cells. Our data show that beta cell-specific CD8 T cells infiltrating the pancreas, the source of antigen, acquire a terminally differentiated multipotency score and are methylated at gene loci related to T-cell stemness such as Tcf7. These results suggest that beta cell-specific CD8 T cells have the capacity to acquire an epigenetic program associated with restricted fate-potential, and raise the possibility that therapeutic approaches that promote tolerance can be reinforced by epigenetic mechanisms. Further supporting the link between the mechanisms that preserve T cell stemness and pancreatic islet destruction, a recent publication describes that the rate of T1D disease progression is inversely related to the establishment of an exhaustion phenotype among beta cell-specific CD8 T cells³⁴. To attenuate the harmful nature of the beta cell-specific CD8 T cells, significant efforts have been mounted to develop new therapeutic strategies to induce tolerance among beta cell-specific CD8 T cells. One such strategy utilized in a recent clinical trial (AbATE) attempted to therapeutically induce a tolerized state among T cells by treating individuals with an anti-CD3 monoclonal antibody. While some patients in this trial exhibited a partial response, the delay in disease progression was ultimately transient^{35,36} Similar to the AbATE study, anti-CD3 treatment administered to at-risk individuals prior to the onset of T1D was also shown to delay disease progression³⁷. These data further reinforce the idea that the initial development of beta cell-specific CD8 T cells impart the cells with a capacity to evade a tolerizing fate³⁸. Therefore, as opposed to generally targeting tolerization of established self-reactive T cells, the epigenetic data from beta cell-specific CD8 T cells that we present here suggest a more successful strategy may involve inducing a tolerized state through an epigenetic reprogramming of the stem-like subset of the autoreactive T cells. Further, our results also highlight the potential utility of using epigenetic programs to track, even predict, the tolerized status of the T cells. Thus, development of the methylation-based T cell multipotency index may serve as a diagnostic tool for assessing long-term tolerance induction.

In the current study, our results reveal that beta cell-specific CD8 T cells acquire epigenetic programs that are coupled to homeostasis and developmental plasticity. Because CD8 T cells resist acquisition of DNA methylation programs found in terminally differentiated T cells, these data raise the possibility that the antigen-specific cells found in circulation (and likely other anatomical locations as well) may provide a pool of self-renewing stem-like cells that can maintain the effector response. Collectively, these data help reconcile the seemingly discordant naïve-like phenotype of beta cell-specific CD8 T cells with results from other studies that document the effector functionality (e.g., expression of granzymes and perforin) of self-reactive CD8 T cells. Beyond our investigation into the disease etiology of T1D, the novel bioinformatic tools that we have established to analyze beta cell-specific T cells has broad implications for understanding the role of methylation programming in T cell differentiation and establishment of long-lived memory T cells. While prior studies examining changes in histone modifications during in vitro memory differentiation of murine CD8 T cells have documented a lineage relationship between naïve and memory CD8 T cells³⁹, here we have established an epigenetic atlas based on DNA methylation of the endogenous human T cell subsets. Our DNA methylation-based T cell multipotency index provides further documentation that progressive differentiation of human T cells is coupled to changes in DNA methylation. By using PD-1+ HIV-specific CD8 T cells from a well-defined cohort of long-term infected individuals, we were able to better define the spectrum of epigenetic reprogramming events that result in the progressive loss of T cell developmental plasticity in humans. In addition to expanding our understanding of human memory T cell differentiation, this multipotency index can be applied toward improving T cell-based therapeutic approaches. As the field moves forward with development of novel strategies to induce tolerance among self-reactive T cells, the epigenetic atlas of T cell differentiation provided here will serve as a framework for assessing the long-lived impact these tolerance-focused strategies have on differentiation status of CD8 T cells. In addition to utilizing this atlas to assess T cell tolerance, the multipotency index provided herein may also serve more broadly as a roadmap for studies that aim to stably modify T cell function for an array of therapeutic purposes.

### Materials and Methods

Isolation of Human CD8 T Cells:

This study was conducted with approval from the Institutional Review Boards (IRBs) of St. Jude Children's Research Hospital, Benaroya Institute (BRI), University of San Francisco (UCSF), and Case Western Reserve University (CWRU). The BRI Registry and Repository Sample Bank ID is IRB07109. For healthy adult donors, PBMCs were collected through the St. Jude Blood Bank, and samples for WGBS were collected under IRB protocol XPD15-086. PBMCs were purified from a platelet-apheresis blood unit using a density gradient. In brief, blood was diluted 1:2.5 using sterile Dulbecco's PBS (Thermo Fisher Scientific). The diluted blood was then overlayed with Ficoll-Paque PLUS medium (GE Healthcare) to a final dilution of 1:2.5 (Ficoll/diluted blood). The gradient was centrifuged at 400×g with no brake for 20 min at room temperature. The PBMC interphase layer was collected, washed with 2% FBS/1 mM EDTA PBS buffer, and centrifuged at 400×g for 5 min. Total CD8 T cells were enriched from PBMCs with the EasySep human CD8 negative-selection kit (STEMCELL Technologies). All donors provided informed consent for collection of the blood samples used for all analyses. Cryopreserved PBMC samples were obtained from HIV-infected participants enrolled in the San Francisco-based SCOPE cohort who were infected for at least two years and on suppressive ART for at least two years with HIV viral load <40 copies/mL. In case of T1D and HIV patients, the same approach has been used to collect PBMCs then tetramer/pentamer staining was performed to identify beta cell-specific and HIV-specific CD8 T cells.

Isolation and Flow Cytometric Analysis of Naive and Memory CD8 T Cell Subsets:

After enrichment of CD8 T cells, naive and memory CD8 T cell subsets were sorted using the following markers, as previously described (L. Gattinoni, et al. Nat Med 17.10 (2011): 1290-1297; E. Lugli et al. Nat. Protoc. 8.1 (2013): 33-42). Naive CD8 T cells were phenotyped as live CD8+, CCR7⁺, CD45RO⁻, CD45RA⁺, and CD95⁻ cells. CD8 Tem cells were phenotyped as live CD8+, CCR7⁻, and CD45RO⁺ cells. TCM cells were phenotyped as live CD8+, CCR7⁺, and CD45RO⁺ cells. Tscm cells were phenotyped as live CD8+, CCR7⁺, CD45RO⁻, and CD95⁺ cells. Sorted cells were checked for purity (i.e., samples were considered pure if >90% of the cells had the desired phenotype). HIV-specific CD8+ T cells were identified by staining with MHC Class I tetramers (made in the laboratory of RPS) or pentamers (Proimmune, Oxford, United Kingdom) and sorted on a FACS Aria.

HLA-A2 Tetramer Assembly:

Peptides representing previously characterized immunodominant beta cell epitopes INS B 10-18, PPI 15-24, GAD 114-122, IA2 797-805, IGRP 265-273, ZnT8 186-494 were synthesised by Genscript (Piscataway, N.J., USA). FILA-A2 monomers (refolded to contain each peptide of interest) were obtained through the National Institutes of Health (NIH) Tetramer Core Facility (Atlanta, Ga., USA). PE and APC multimers were prepared utilizing a stepwise addition of fluophore labeled streptavidin slightly modified from the NIH Tetramer Core protocol. Briefly, 60 μl of each monomer (0.2 μg/μl) diluted 1:10 in PBS was multimerised by adding 1.2 μl of the appropriate PE or APC-labelled streptavidin 0.2 mg/ml (Thermo Fisher Scientific, Waltham, Mass., USA) for 10 mill at 4° C. 6 times. Multimers were stored at 4° C. and used within 4 weeks.

Ex Vivo CD8 T-Cell Sorting:

Samples from HLA-A2-positive subjects were dual stained with a mixture containing a pool of PE and APC tetramers, PBMCs (20×10\hat{ }6) were stained simultaneously with 3 μl each in 200 μl of PBS supplemented with 2% BSA and incubated for 15 min at 37° C. Cells were then incubated with 30 μl each of anti-PE and anti-APC magnetic beads (Miltenyi Biotec, San Diego, Calif., USA) and enriched on a MS-sized magnetic column according to the manufacturer's instructions. After enrichment, flowthrough cells were reserved for bulk memory and naïve CD8 T-cell sorting described in the following section. Both enriched and flowthrough cells samples were washed and then stained for 30 min at 4° C. with 5 μl each: anti-CD8 BV605 (Biolegend.), AF700 CD45RA (BD Bioscience), APC-Cy7 CCR7 (Biolegend), PerCP-eFluor 710 TIGIT (eBioscience), PE-Cy7 PD-1(Biolegend), BV650 CD95 (Biolegend), BV421 KLRG1 (Biolegend), 2.5 μl each of anti-CD4/CD14/CD16/CD20/CD40-FITC (dump channel; eBioscience, Waltham, Mass., USA), and 1:10,000 syTOX green viability dye (Thermo Fisher). CD8 cells were sorted using a FACSAria H (BD Biosciences) into lysis buffer from an Allprep DNA RNA mini prep kit (Qiagen) and processed for DNA and RNA according to the manufacturer's instructions.

In Vitro CD8 T Cell Ex Vivo Expansion:

Isolated PBMCs were labeled with Cell Trace Violet-CTV (Life Technologies) at a final concentration of 104. CTV-labeled cells were maintained in culture in RPMI containing 10% FBS, penicillin-streptomycin containing mixture of 6 pooled peptides at a final concentration total of 100 ug/ml. After 14-24d of incubation at 37° C. and 5% CO₂, adding medium and interleukin (IL)-2 as needed starting on day 7 cultures were collected for sorting. To visualize responses, cells were stained with tetramers and surface markers as described previously, and undivided (CTV++) and divided cells(CTV+/−) tetramer+ CD8 T-cells were sorted into lysis buffer. To define the phenotypic characteristic features of the expanded cells, we used cell surface markers CD95, CCR7, and CD45RA.

Isolation and Phenotypic Analysis of Mouse Antigen-Specific CD8 T Cells:

We adoptively transferred 2000 congenically distinct naive P14 CD8 T cells (CD45.1/1+) into C57BL/6 mice (CD45.1/2+). One day later, we infected the mice with different strains of lymphocytic choriomeningitis virus (LCMV) separately. Acute LCMV infection was performed by i.p. injection of 2×10\hat{ }5 PFU Armstrong strain per mouse, while chronic LCMV infections were performed by i.v. injection of 2×10\hat{ }6 PFU LCMV per mouse using either Clone 13 strain or C6 strain that has a mutated GP33 epitope with lower TCR-binding affinity to P14 cells (D. T. Utzschneider et al., et al. J Exp Med. 213.9 (2016): 1819-1834). After 4 or 8 weeks of LCMV infections we harvested the spleens and FACS-purified P14 cells from splenocytes. P14 cells were also phenotypically analyzed by Flow Cytometry after surface staining using monoclonal antibodies for CD45.1 (clone A20), CD45.2 (clone 104), CD8 (clone 53-6.7), PD-1(clone J43), and Klrg1 (clone 2F1).

Genomic Methylation Analysis:

DNA was extracted from the sorted cells by using a DNA-extraction kit (QIAGEN) and then bisulfite treated using an EZ DNA methylation kit (Zymo Research), which converts all unmethylated cytosines to uracils. Loci-specific PCR was performed using the primers described below. Individual clones of DNA were sequenced using the previously described blue/white bacterial cloning and screening approach (Abdelsamed et al., J Exp Med 214.6 (2017): 1593-1606). Whole geneome bisulfite sequencing was performed as previously described. Briefly, bisulfite modified DNA-sequencing libraries were generated using the EpiGnome kit (Epicentre) per the manufacturer's instructions. Bisulfite-modified DNA libraries were sequenced using an Illumina HiSeq system (Abdelsamed et al., J Exp Med 214.6 (2017): 1593-1606). Sequencing data were aligned to the HG19 genome by using BSMAP soft-ware (Y. Xi, W. Li. BMC Bioinformatics. 10.1 (2009): 232). Differentially methylation analysis of CpG methylation among the datasets was determined with a Bayesian hierarchical model to detect regional methylation differences with at least three CpG sites (Wu et al., Nucleic Acids Res. 43.21 (2015): e141). To perform loci-specific methylation analysis, bisulfite modified DNA was PCR amplified with locus-specific primers. The PCR amplicon was cloned into a pGEMT easy vector (Promega) and then transformed into XL10-Gold ultracompetent bacteria (Agilent Technologies). Bacterial colonies were selected using a blue/white X-gal selection system after overnight growth, the cloning vector was then purified from individual colonies, and the genomic insert was sequenced. After bisulfite treatment, the methylated CpGs were detected as cytosines in the sequence, and unmethylated CpGs were detected as thymines in the sequence by using QUMA software (Kumaki et al., Nucleic Acids Res. 36 (2008): W170-W175).

### Human and Mouse Primer Sequences for Loci Specific Bisulfite Sequencing:

Forward Human TOX: 5′-AGTAAGGTTTTTTTTTAACAATAGG-3′,
Reverse Human TOX: 5′-CAATAAAATCATTCTAAAAAATAACAAC-
3′
Forward Human DNMT3A: GAAGGTGTATTGAAGTGTGG-3′
Reverse Human DNMT3A: 5′-CCAAAAAAAAAACCCAACCCA-3′
Forward Mouse TOX: 5′-GTGTAAGTTATTGTGATTCTGATTGTG-3′
Reverse Mouse TOX: 5′-
CTTTAACTACCCTCTCTAAATTAAAAAACC-3′
Forward Mouse DNMT3A: 5′-GGTTTTTGGATAGAGTGGGGATA-3′
Reverse Mouse DNMT3A: 5-CAAAAACTACCAAACVCATCAAACC-3′

T-Cell Multipotency Index:

To identify the methylation state of the CpG sites associated with the T cell multipotent potential, a supervised analysis was performed between the methylomes from two naïve and four HIV-specific CD8 T cells (methylation difference>=0.4 and FDR<=0.01). This analysis results in identification of 245 CpGs sites that were hypomethylated in naïve HIV CD8 T cells compared to HIV-specific CD8 T cells. Each set of the CpGs was then used as an input to the one-class logistic regression to calculate the multipotency signature using just the HIV naive samples (Training data sets). Once the signature was obtained, it was then applied to the beta cell-specific, HIV-specific, Naïve, Tscm, Tem, and Tcm CD8 T cell methylomes (Test data sets) (T. M. Malta, et al. Cell. 173.2 (2018): 338-354; A. Sokolov, et al. PLoS Comput Biol. 12.3 (2016): e1004790). The score was calculated as the dot product between the DNA methylation value and the signature. The score was subsequently converted to the [0, 1] range. Data sets with multipotency indices closer to 1 were more similar to naïve cells.

Isolation of Murine Beta Cell-Specific CD8 T Cells from NOD Mice:

Female, NOD mice, 8-14 weeks of age were euthanized by CO₂ asphyxiation, followed by cervical dislocation. Prior to euthanasia, urine glucose was tested to exclude diabetic mice. Cells from the pancreas were harvested by perfusing the pancreas with 3 ml of collagenase IV (Gibco 17104019) (410 units/ml in Hanks' balanced salt solution (HBSS) and 10% fetal bovine serum (FBS)) into the common bile duct using a 30G needle. The excised pancreas was incubated for 20 minutes at 37° C. in 3-5 ml of collagenase, passed through a 40 um cell strainer, and centrifuged at 220 rcf for 3 minutes. Cells were then washed twice in 10 ml of HBSS+10% FBS and centrifuged at 600 rcf for 3 minutes and resuspended in complete RPMI. For sorting, cells were washed in PBS twice and stained with the following antibodies: KLRG1 FITC (2F1; eBioscience 11-5893-82), CD62L Percp-cy5.5 (MEL-14; eBioscience 45-0621-82), CD8a PE-cy7 (53-6.7; BioLegend 100722), CD127 BV421 (A7R34; BioLegend 135024), Viability-Zombie Aqua (BioLegend 423101), CD44 APC (IM7; eBioscience 17-0441-82), CD4 BV711 (RM4-5; BioLegend 100550), PD-1 APC-750/Fire (29F.1A12; BioLegend 135240), and mouse NRP-V7 mimotope tetramer PE (KYNKANVFL/H-2K^{d}; NIH tetramer facility)³⁰. Cells were sorted on viability dye-negative, CD4⁻CD8⁺, and tetramer⁺CD44⁺ on a Sony ICyt Synergy (Sony Biotechnology, Inc.). DNA was extracted from FACS purified cells and used for whole-genome DNA methylation profiling as described above.

Single Cell ATAC (Assay for Transposase Accessible Chromatin) Seq:

Nuclei were isolated from FACS purified tetramer+ beta cell-specific CD8 T cells and polyclonal Tem and naïve CD8 T cells from T1D donors using the Low Cell Input Nuclei Isolation protocol from 10x Genomics. The transposition reaction was then performed on the bulk nuclei. The transposed nuclei were partitioned into nanoliter-scale barcoded Gel Beads-in-emulsion (GEMs) after running the loaded Chromium microfluidic Chip E on the Chromium Controller, and the transposed DNA was uniquely indexed and barcoded for each individual nucleus per manufacturer's instructions (10x Genomics). Libraries were generated and sequenced using Illumina Hiseq system following the manufacturer's protocols. For each sample, the single cell ATAC data were mapped to hg38 using the Cell Ranger ATAC pipeline. We then used SnapATAC (https://github.com/r3fang/SnapATAC) to combine multiple samples, i.e. the fragment.tsv files generated by Cell Ranger ATAC pipeline, and performed the clustering analysis.

Code Availability:

All custom computational code will be made available upon request.

TABLE 1
T cell multipotency index weight score

| Chromosomal location of CpG site | weight score |
|---|---|
| chr1*54763966*54763967 | −0.051489205 |
| chr1*54763970*54763971 | −0.040885201 |
| chr1*200842959*200842960 | −0.032164403 |
| chr1*200842973*200842974 | −0.037013444 |
| chr1*200842975*200842976 | −0.041142467 |
| chr1*200842985*200842986 | −0.030448708 |
| chr1*200843055*200843056 | −0.032573919 |
| chr1*200843153*200843154 | −0.020320909 |
| chr1*200843155*200843156 | −0.029571821 |
| chr11*67186981*67186982 | −0.03624503 |
| chr11*67187005*67187006 | −0.03766482 |
| chr14*24601827*24601828 | −0.002907005 |
| chr17*1962822*1962823 | −0.014098444 |
| chr17*75428521*75428522 | −0.051639602 |
| chr2*128397880*128397881 | −0.022278802 |
| chr2*128397886*128397887 | −0.026131514 |
| chr3*195677892*195677893 | −0.025391733 |
| chr6*43109697*43109698 | −0.046761064 |
| chr7*1858913*1858914 | −0.034032727 |
| chr7*1858915*1858916 | −0.031290377 |
| chr7*2550728*2550729 | −0.037210104 |
| chr7*5528338*5528339 | −0.050665893 |
| chr7*5528344*5528345 | −0.05077263 |
| chr7*5528350*5528351 | −0.05328968 |
| chr8*41465767*41465768 | 0.014817496 |
| chr8*144599224*144599225 | 0.000554266 |
| chr17*1508226*1508227 | −0.052542485 |
| chr3*195677889*195677890 | −0.023793919 |
| chr17*1962819*1962820 | −0.040145784 |
| chr1*200843206*200843207 | −0.037724395 |
| chr8*144599149*144599150 | −0.048410743 |
| chr14*24601657*24601658 | −0.020068353 |
| chr3*195677852*195677853 | −0.035095745 |
| chr5*79494331*79494332 | −0.046329321 |
| chr14*91793433*91793434 | −0.075230935 |
| chr14*24601869*24601870 | −0.028575902 |
| chr8*144599138*144599139 | −0.036880663 |
| chr9*131831540*131831541 | −0.039731515 |
| chr14*24601674*24601675 | −0.010176824 |
| chr2*240319164*240319165 | −0.02842066 |
| chr1*200843190*200843191 | −0.038845847 |
| chr3*128825796*128825797 | −0.050389701 |
| chr9*132096906*132096907 | −0.037161201 |
| chr1*200843199*200843200 | −0.016741001 |
| chr22*19873233*19873234 | −0.033627352 |
| chr22*19873238*19873239 | −0.032581322 |
| chr2*240319143*240319144 | −0.021739709 |
| chr5*79494345*79494346 | −0.016968116 |
| chr5*79494329*79494330 | −0.031237907 |
| chr1*47899390*47899391 | −0.03029286 |
| chr19*17413672*17413673 | 0.00035382 |
| chr17*1962794*1962795 | −0.035931787 |
| chr3*128825767*128825768 | −0.015163589 |
| chr9*132096893*132096894 | −0.050484434 |
| chr11*1778617*1778618 | −0.043686951 |
| chr8*41465751*41465752 | −0.030823525 |
| chr8*41465738*41465739 | −0.038911002 |
| chr8*41465740*41465741 | −0.026517593 |
| chr19*47990271*47990272 | −0.045511306 |
| chr22*19873255*19873256 | −0.024893427 |
| chr14*24601654*24601655 | −0.028220629 |
| chr19*47990273*47990274 | −0.030124126 |
| chr19*18775108*18775109 | −0.062101468 |
| chr14*24601641*24601642 | −0.016301541 |
| chr16*2286811*2286812 | −0.01462102 |
| chr16*2286813*2286814 | −0.006753333 |
| chr1*47899517*47899518 | −0.029880533 |
| chr19*17413755*17413756 | −0.016336294 |
| chr14*24601687*24601688 | −0.014617311 |
| chr5*79494377*79494378 | −0.021020368 |
| chr14*24601807*24601808 | −0.032839493 |
| chr3*186543457*186543458 | −0.034245319 |
| chr16*1586266*1586267 | −0.045254966 |
| chr14*24601503*24601504 | −0.020196436 |
| chr1*34502915*34502916 | −0.018537915 |
| chr17*1508203*1508204 | −0.063149342 |
| chr12*124873717*124873718 | −0.036865441 |
| chr3*186543460*186543461 | −0.016954625 |
| chr9*134139875*134139876 | −0.034745302 |
| chr16*88837466*88837467 | −0.031977782 |
| chr9*131831537*131831538 | −0.033216512 |
| chr5*79494382*79494383 | −0.022330592 |
| chr19*17413736*17413737 | −0.021118566 |
| chr17*7381209*7381210 | −0.034781965 |
| chr14*91793740*91793741 | −0.052651011 |
| chr19*17413747*17413748 | −0.028457434 |
| chr12*124873721*124873722 | −0.026676099 |
| chr5*149511628*149511629 | −0.013789182 |
| chr11*100998839*100998840 | −0.009439961 |
| chr3*73673729*73673730 | −0.012548735 |
| chr9*134139853*134139854 | −0.043737883 |
| chr10*73849625*73849626 | −0.037355053 |
| chr5*149511630*149511631 | −0.025314631 |
| chr15*41062821*41062822 | −0.035660499 |
| chr3*73673727*73673728 | −0.024387665 |
| chr14*95929088*95929089 | −0.038143505 |
| chr1*34502912*34502913 | −0.02550095 |
| chr9*134139877*134139878 | −0.028128843 |
| chr19*17413733*17413734 | −0.034525355 |
| chr6*48036203*48036204 | −0.021686697 |
| chr5*133449482*133449483 | −0.030453934 |
| chr16*57176270*57176271 | −0.035743317 |
| chr16*1586282*1586283 | −0.037115103 |
| chr19*47115298*47115299 | −0.020487134 |
| chr6*14275750*14275751 | −0.035043126 |
| chr6*48036192*48036193 | −0.018058932 |
| chr5*133449478*133449479 | −0.021259607 |
| chr6*48036211*48036212 | −0.023207556 |
| chr6*48036213*48036214 | −0.018295687 |
| chr19*47115301*47115302 | −0.026002811 |
| chr11*47177341*47177342 | −0.043659776 |
| chr19*17413751*17413752 | −0.025152814 |
| chr11*47177337*47177338 | −0.041577936 |
| chr6*43109701*43109702 | −0.067065772 |
| chr15*70850910*70850911 | −0.056708938 |
| chr15*41062774*41062775 | −0.030287613 |
| chr2*10441826*10441827 | −0.024253009 |
| chr11*94770665*94770666 | −0.019000793 |
| chr11*66083771*66083772 | −0.04024361 |
| chr1*244213467*244213468 | −0.027703043 |
| chr2*73277501*73277502 | −0.035312451 |
| chr11*94770663*94770664 | −0.006697882 |
| chr5*9545638*9545639 | −0.017612105 |
| chr11*100998723*100998724 | −0.015476251 |
| chr2*73277504*73277505 | −0.029954833 |
| chr11*100998715*100998716 | −0.029312448 |
| chr6*43109948*43109949 | −0.037927571 |
| chr11*94770655*94770656 | −0.012510686 |
| chr16*87370995*87370996 | −0.027354052 |
| chr17*7742028*7742029 | −0.034562731 |
| chr17*76120865*76120866 | −0.014332076 |
| chr17*55678832*55678833 | −0.016840275 |
| chr17*76120798*76120799 | −0.035465594 |
| chr11*100998717*100998718 | −0.019927946 |
| chr17*76120803*76120804 | −0.02908226 |
| chr14*24601498*24601499 | −0.021622346 |
| chr17*55678980*55678981 | −0.040666105 |
| chr5*9545636*9545637 | −0.007406856 |
| chr1*2164005*2164006 | −0.039536171 |
| chr19*10824144*10824145 | −0.038182949 |
| chr21*43954568*43954569 | −0.039248949 |
| chr8*27468476*27468477 | −0.030810656 |
| chr10*51504040*51504041 | −0.024637151 |
| chr17*55678922*55678923 | −0.025588 |
| chr21*43954573*43954574 | −0.031210915 |
| chr12*125219046*125219047 | −0.032792728 |
| chr12*125219073*125219074 | −0.042626273 |
| chr11*66083780*66083781 | −0.016053967 |
| chr11*66083765*66083766 | −0.029634226 |
| chr17*76120808*76120809 | −0.026847212 |
| chr16*30817689*30817690 | −0.016057633 |
| chr4*26031260*26031261 | −0.042137501 |
| chr9*7246299*7246300 | −0.036370623 |
| chr16*88764823*88764824 | −0.050824787 |
| chr19*17221198*17221199 | −0.012055646 |
| chr16*30817712*30817713 | −0.037189744 |
| chr1*26098054*26098055 | −0.037150512 |
| chr17*76120788*76120789 | −0.023087521 |
| chr17*38104127*38104128 | −0.025967651 |
| chr1*229272781*229272782 | −0.020546754 |
| chr17*55678927*55678928 | −0.022204518 |
| chr6*149772306*149772307 | −0.03605673 |
| chr9*140357145*140357146 | −0.041180756 |
| chr19*56989493*56989494 | −0.032102914 |
| chr16*88764686*88764687 | −0.01432883 |
| chr5*175085912*175085913 | −0.023535101 |
| chr21*47309230*47309231 | −0.042839927 |
| chr2*63280219*63280220 | −0.021922079 |
| chr1*20085777*20085778 | −0.023608178 |
| chr21*43818498*43818499 | −0.044253868 |
| chr1*153670607*153670608 | −0.028551329 |
| chr17*75429510*75429511 | −0.035644752 |
| chr13*40762149*40762150 | −0.024023642 |
| chr12*110384505*110384506 | −0.028526075 |
| chr16*57567602*57567603 | −0.03387585 |
| chr15*40395576*40395577 | −0.026255473 |
| chr22*38025181*38025182 | −0.031937112 |
| chr16*89615493*89615494 | −0.024395071 |
| chr3*42262026*42262027 | −0.025167533 |
| chr5*6766879*6766880 | −0.030106999 |
| chr17*43221326*43221327 | −0.021207909 |
| chr10*48573260*48573261 | −0.031646834 |
| chr19*30107417*30107418 | −0.083689017 |
| chr3*195627549*195627550 | −0.031079222 |
| chr6*15327816*15327817 | −0.020234644 |
| chr7*32467700*32467701 | −0.024652316 |
| chr4*1321148*1321149 | −0.043021483 |
| chr15*75402067*75402068 | −0.026523455 |
| chr9*37033917*37033918 | −0.024063525 |
| chr7*5527947*5527948 | −0.030270429 |
| chr5*140221051*140221052 | −0.022633822 |
| chr17*74476786*74476787 | −0.024172254 |
| chr2*43445306*43445307 | −0.033274704 |
| chr19*18416902*18416903 | −0.018575725 |
| chr7*2124990*2124991 | −0.035829663 |
| chr22*24554542*24554543 | −0.035811893 |
| chr12*122610165*122610166 | −0.026095695 |
| chr16*11234502*11234503 | −0.028371946 |
| chr9*37576000*37576001 | −0.024005964 |
| chr4*1323220*1323221 | −0.031967203 |
| chr2*157179537*157179538 | −0.036153963 |
| chr19*18810916*18810917 | −0.031544982 |
| chr1*19250206*19250207 | −0.051102026 |
| chr16*85617370*85617371 | −0.025074542 |
| chr7*149565728*149565729 | −0.021569223 |
| chr18*77190165*77190166 | −0.037854589 |
| chr11*117694358*117694359 | −0.021555763 |
| chr7*949634*949635 | −0.032992718 |
| chr11*2320456*2320457 | −0.021311307 |
| chr4*2403929*2403930 | −0.017038098 |
| chr5*1103732*1103733 | −0.035286956 |
| chr2*197013974*197013975 | −0.046064225 |
| chr5*454403*454404 | −0.022011673 |
| chr9*130797344*130797345 | −0.015604099 |
| chr13*50707638*50707639 | −0.018621312 |
| chr1*31225357*31225358 | −0.033053702 |
| chr18*57585759*57585760 | −0.028704362 |
| chr1*235757109*235757110 | −0.034559815 |
| chr6*7196327*7196328 | −0.022646244 |
| chr20*61524519*61524520 | −0.021071566 |
| chr7*1964189*1964190 | −0.004542149 |
| chr22*44464535*44464536 | −0.0264052 |
| chr5*141703565*141703566 | −0.026724318 |
| chr16*89381257*89381258 | −0.021951965 |
| chr1*9378058*9378059 | −0.022433093 |
| chr11*128337079*128337080 | −0.021776016 |
| chr11*76839475*76839476 | −0.022046699 |
| chr8*134507964*134507965 | −0.037368803 |
| chr2*9901313*9901314 | −0.035644791 |
| chr19*54446099*54446100 | −0.013361954 |
| chr2*229046227*229046228 | −0.019541245 |
| chr19*55553217*55553218 | −0.01519386 |
| chr6*155485691*155485692 | −0.012613361 |
| chr5*140573677*140573678 | −0.019893385 |
| chr19*18612953*18612954 | −0.019858269 |
| chr6*37503745*37503746 | −0.044785912 |
| chr2*133104908*133104909 | −0.022013299 |
| chr1*47898434*47898435 | −0.020770104 |
| chr19*47324527*47324528 | −0.029441027 |
| chr2*45173253*45173254 | −0.010230763 |
| chr3*126006283*126006284 | −0.018008457 |
| chr2*233186258*233186259 | −0.025748517 |
| chr17*50236034*50236035 | −0.013432358 |
| chr16*14097271*14097272 | −0.018283733 |
| chr3*46607215*46607216 | −0.031347416 |

We claim:

1. A method of identifying the stage of differentiation of subject T cells, said method comprising:
d) measuring the methylation status of the subject T cells;
e) establishing a multipotency score for the subject T cells based on a comparison of the methylation status of the subject T cells to a T-cell multipotency index;
f) identifying the stage of differentiation of the subject T cells based on the multipotency score.

2. The method of claim 1, wherein the stage of differentiation is naïve T cells, stem memory T cells, self-reactive T cells, central memory T cells, or effector memory T cells.

3. The method of any one of claims 1-3, wherein the measurement step comprises measuring the methylation status of one or more CpG site in the subject T cells.

4. The method of claim 3, wherein the one or more CpG sites comprise one or more of the CpG sites selected from Table 1.

5. The method of claim 4, wherein the one or more CpG sites comprise two or more of the CpG sites selected from Table 1.

6. The method of any one of claims 1-3, wherein the measurement step comprises measuring the methylation status of each of the 245 CpG sites in Table 1.

7. The method of any one of claims 1-6, further comprising generating a multipotency index by identifying CpG sites that are differentially methylated in at least two populations of T cells and assigning a weighted index score to each CpG site.

8. The method of claim 7, wherein the CpG sites are identified by supervised analysis of training data sets comprising genome-wide methylation profiles of the least two populations of T cells, wherein the at least two populations of T cells are at different stages of differentiation.

9. The method of claim 8, where the T cells are selected from naïve T cells, stem memory T cells, central memory T cells, effector memory T cells, or effector memory-like T cells.

10. The method of claim 9, wherein the effector memory-like T cells are HIV-specific T cells.

11. The method of claim 10, wherein the at least two populations of T cells comprise one or more populations of naïve T cells and one or more populations of HIV-specific T cells.

12. The method of claim 9, wherein the at least two populations of T cells comprise one or more populations of naïve T cells and one or more populations of effector memory T cells.

13. The method of any one of claims 7-12, wherein one or more machine learning algorithm identifies the differentially methylated CpG sites and generates weighted index scores for each identified CpG site, thereby generating the multipotency index.

14. The method of claim 13, wherein the one or more machine learning algorithm is a one-class logistic regression algorithm.

15. The method of any one of claims 1-14, wherein the T cell multipotency index comprises one or more weighted index scores, wherein each weighted index score corresponds to a CpG site in a T cell genome.

16. The method of claim 15, wherein the step of establishing the multipotency score comprises determining the dot product between the methylation status of the subject T cells at the one or more CpG sites and the corresponding weighted index score for the CpG site.

17. The method of claim 15 or 16, wherein the weighted score corresponds to a CpG site selected from Table 1.

18. The method of claim 17, wherein the step of establishing the multipotency score comprises determining the dot product between the methylation status of the subject T cells at one or more CpG sites selected from Table 1 and the corresponding weighted index score in Table 1.

19. The method of claim 18, wherein the step of establishing the multipotency score comprises determining the dot product between the methylation status of the subject T cells at two or more CpG sites selected from Table 1 and the corresponding weighted index score in Table 1.

20. The method of claim 19, wherein the step of establishing the multipotency score comprises determining the dot product between the methylation status of the subject T cells at each CpG site selected from Table 1 and the corresponding weighted index score in Table 1.

21. The method of claim 1-20, wherein the multipotency score is a normalized multipotency score.

22. The method of claim 21, wherein the step of establishing the multipotency score comprises normalizing the multipotency score to a range of 0 to 1.

23. The method of claim 22, wherein a normalized multipotency score above 0.1, 0.2, 0.3, 0.4, 0.5, 0.6, 0.7, 0.8, or 0.9 indicates the subject T cells have a high differentiation potential and a score below 0.9, 0.8, 0.7, 0.6, 0.5, 0.4, 0.3, 0.2, or 0.1 indicates the subject T cells have a low differentiation potential.

24. The method of any one of claims 1-23, wherein a multipotency score higher than a control evaluated by the same method identifies the T cell as having increased differentiation potential relative to the control.

25. The method of any one of claims 1-23, wherein a multipotency score higher than a pre-established threshold score identifies the T cell as having increased differentiation potential.

26. The method of any one of claims 1-25, wherein the subject T cell is a CD8 T cell.

27. The method of claim 26, wherein the CD8 T cell is a human CD8 T cell.

28. The method of any one of claims 1-27, wherein the subject T cell is collected from a patient.

29. The method of claim 28, wherein the patient has cancer, an autoimmune disease, or a chronic infection.

30. The method of claim 29, wherein the autoimmune disease is type-1 diabetes.

31. The method of claim 29 or 30, wherein the method is effective to identify tolerance induction among the T cells collected from the patient.

32. The method of claim 31, wherein the T cells are self-reactive T cells.

33. The method of claim 32, wherein the T cells are beta cell-specific CD8 T cells.

34. The method of any one of claims 29-33, wherein the patient has been previously administered a therapeutic that induces T cell tolerance.

35. A method of isolating a population of T cells with improved differentiation potential, said method comprising:
f) dividing a starting population of T cells into at least three subpopulations;
g) measuring the methylation status of the T cells in each subpopulation;
h) establishing a multipotency score based on a comparison of the methylation status to a T cell multipotency index;
i) identifying subpopulations of T cells having increased differentiation potential based on the multipotency score; and
j) combining at least two identified subpopulations of T cells having increased differentiation potential into a final population of T cells, wherein at least one subpopulation of the starting population of T cells is not combined into the final population of T cells.

36. The method of claim 35, wherein the differentiation potential of the final population of T cells is increased relative to the differentiation potential of a natural population of CD8 T cells from the same origin.

37. The method of claim 35, wherein the multipotency score of the final population of T cells is increased relative to a control.

38. The method of claim 35, wherein the multipotency score of the final population of T cells is increased relative to a pre-defined threshold.

39. The method of any one of claims 35-38, wherein the measurement step comprises measuring the methylation status of one or more CpG sites in the T cells.

40. The method of claim 39, wherein the one or more CpG sites comprise one or more of the CpG sites selected from Table 1.

41. The method of claim 40, wherein the one or more CpG sites comprise two or more of the CpG sites selected from Table 1.

42. The method of any one of claims 35-38, wherein the measurement step comprises measuring the methylation status of at least 5%, at least 10%, at least 20%, at least 30%, at least 40%, at least 50%, at least 60%, at least 70%, at least 80%, or at least 90% of the 245 CpG sites in Table 1.

43. The method of any one of claims 35-38, wherein the measurement step comprises measuring the methylation status of each of the 245 CpG sites in Table 1.

44. The method of any one of claims 35-43, further comprising generating a multipotency index by identifying CpG sites that are differentially methylated in the at least two populations of T cells and assigning a weighted index score to each CpG site.

45. The method of claim 44, wherein the CpG sites are identified by supervised analysis of training data sets comprising genome-wide methylation profiles of the least two populations of T cells, wherein the at least two populations of T cells are at different stages of differentiation.

46. The method of claim 45, where the T cells are selected from naïve T cells, stem memory T cells, central memory T cells, effector memory T cells, or effector memory-like T cells.

47. The method of claim 46, wherein the effector memory-like T cells are HIV-specific CD8 T cells.

48. The method of claim 45, wherein the at least two populations of T cells comprise one or more populations of naïve T cells and one or more populations of HIV-specific T cells.

49. The method of claim 45, wherein the at least two populations of T cells comprise one or more populations of naïve T cells and one or more populations of effector memory T cells.

50. The method of any one of claims 44-49, wherein one or more machine learning algorithms identifies the differentially methylated CpG sites and generates weighted index scores for each identified CpG site, thereby generating the multipotency index.

51. The method of claim 50, wherein the one or more machine learning algorithm is a one-class logistic regression algorithm.

52. The method of any one of claims 35-51, wherein the T cell multipotency index comprises one or more weighted index scores, wherein each weighted index score corresponds to a CpG site in a T cell genome.

53. The method of claim 52, wherein the step of establishing the multipotency score comprises determining the dot product between the methylation status of the subject T cells at the one or more CpG sites and the corresponding weighted index score for the CpG site.

54. The method of claim 52 or 53, wherein the weighted score corresponds to a CpG site selected from Table 1.

55. The method of claim 54, wherein the step of establishing the multipotency score comprises determining the dot product between the methylation status of the subject T cells at one or more CpG sites selected from Table 1 and the corresponding weighted index score in Table 1.

56. The method of claim 55, wherein the step of establishing the multipotency score comprises determining the dot product between the methylation status of the subject T cells at two or more CpG sites selected from Table 1 and the corresponding weighted index score in Table 1.

57. The method of claim 56, wherein the step of establishing the multipotency score comprises determining the dot product between the methylation status of the subject T cells at each CpG site selected from Table 1 and the corresponding weighted index score in Table 1.

58. The method of claim 35-57, wherein the multipotency score is a normalized multipotency score.

59. The method of claim 58, wherein the step of establishing the multipotency score comprises normalizing the multipotency score to a range of 0 to 1.

60. The method of claim 59, wherein a normalized multipotency score above 0.1, 0.2, 0.3, 0.4, 0.5, 0.6, 0.7, 0.8, or 0.9 indicates the subject T cells have high differentiation potential and a score below 0.9, 0.8, 0.7, 0.6, 0.5, 0.4, 0.3, 0.2, or 0.1 indicates the subject T cells have a low differentiation potential.

61. The method of any one of claims 35-60, wherein a multipotency score higher than a control a control identifies the T cells as having increased differentiation potential relative to the control.

62. The method of any one of claims 35-61, wherein a multipotency score higher than a pre-established threshold score identifies the T cell as having increased differentiation potential.

63. The method of any one of claims 35-62, wherein the subject T cell is a CD8 T cell.

64. The method of claim 63, wherein the CD8 T cell is a human CD8 T cell.

65. The method of any one of claims 35-64, wherein cells in the final population of T cells comprise a methylated TOX locus.

66. The method of any one of claims 35-65, wherein cells in the final population of T cells comprise an unmethylated DNMT3a locus.

67. The method of any one of claims 35-66, wherein cells in the final population of T cells comprise a methylated BATF locus.

68. A population of T cells isolated by the method of any one of claims 35-67, wherein the T cells have increased differentiation potential relative to a control.

69. A pharmaceutical composition comprising said population of CD8 T cells of claim 68.

70. The pharmaceutical composition of claim 69, wherein the pharmaceutical composition further comprises a pharmaceutically acceptable carrier.

71. A method of treating a chronic infection, an autoimmune disease, or a cancer in a subject, said method comprising administering to the subject the pharmaceutical composition of claim 69 or 70.

72. A method of monitoring T cell differentiation in a patient having an autoimmune disease, comprising:
e) collecting a sample from the patient containing a population of T cells;
f) measuring the methylation status of the T cells in the sample;
g) establishing a multipotency score for the T cells based on a comparison of the methylation status of the T cells to a T-cell multipotency index;
h) identifying the level of auto-reactive T cells in the sample based on the multipotency score.

73. The method of claim 72, wherein a multipotency score higher than a control indicates a high level of auto-reactive T cells, thereby identifying the patient as one who requires further monitoring or treatment.

74. The method of claim 72, wherein a multipotency score lower than a control indicates a low level of auto-reactive T cells, thereby identifying the patient as one in which T cell tolerance has been induced.

75. The method of claim 73 or 74, wherein the control is a T cell population obtained from the patient at a previous time point.

76. The method of claim 73 or 74, wherein the control is a pre-defined threshold.

77. The method of claim 73 or 74, wherein the control is a T cell population obtained from a healthy individual.

78. The method of any one of claims 72-77, wherein the autoimmune disease is type 1 diabetes.

79. The method of any one of claims 73-78, wherein the patient is one who has previously been administered a therapeutic to treat the autoimmune disease.

80. A method of generating a T cell multipotency index, said method comprising:
d) isolating at least two populations of T cells;
e) identifying CpG sites that are differentially methylated in the at least two populations of T cells; and
f) assigning a weighted index score to each CpG site.

81. The method of claim 80, wherein the CpG sites are identified by supervised analysis of training data sets comprising genome-wide methylation profiles of the least two populations of T cells, wherein the at least two populations of T cells are at different stages of differentiation.

82. The method of claim 81, where the T cells are selected from naïve T cells, stem memory T cells, central memory T cells, effector memory T cells, or effector memory-like T cells.

83. The method of claim 82, wherein the effector memory-like T cells are HIV-specific CD8 T cells.

84. The method of claim 83, wherein the at least two populations of T cells comprise one or more populations of naïve T cells and one or more populations of HIV-specific CD8 T cells.

85. The method of claim 82, wherein the at least two populations of T cells comprise one or more populations of naïve T cells and one or more populations of effector memory cells.

86. The method of any one of claims 80-85, wherein one or more machine learning algorithm identifies the differentially methylated CpG sites and generates weighted index scores for each identified CpG site, thereby generating the multipotency index.

87. The method of claim 86, wherein the one or more machine learning algorithm is a one-class logistic regression algorithm.

88. The method of any one of claims 80-87, wherein the T cell multipotency index comprises one or more weighted index scores, and wherein each weighted index score corresponds to a CpG site in a T cell genome.

89. A kit comprising reagents for detecting a methylation status of one or more CpG sites selected from Table 1 in subject T cells, wherein the kit further includes instructions for accessing, utilizing, or generating a multipotency index.

90. The kit of claim 89, comprising reagents for detecting the methylation status of at least 5%, at least 10%, at least 20%, at least 30%, at least 40%, at least 50%, at least 60%, at least 70%, at least 80%, or at least 90% of the 245 CpG sites in Table 1 in the subject T cells.

91. The kit of claim 89, comprising reagents for detecting the methylation status of each CpG site in Table 1 in the subject T cells.

92. The kit of claim 89, consisting of reagents for detecting the methylation status of each CpG site in Table 1 in the subject T cells.

93. The kit of any one of claims 89-92, further comprising a package insert comprising instructions for accessing, utilizing, or generating a T cell multipotency index based on the methylation status of the one or more CpG sites.
